(19) United States
(12) Patent Application Publication
(10) Pub. No.: US 2011/0257111 A1
(43) Pub. Date: Oct. 20, 2011

(54) Hydroxyethlamino Sulfonamide Derivatives

(76) Inventors: Scott L. Harbeson, Cambridge, MA (US); Carig E. Masse, Cambridge, MA (US)

(21) Appl. No.: 13/125,464

(22) PCT Filed: Oct. 23, 2009
(86) PCT No.: PCT/US09/05773

§ 371 Date: Jul. 5, 2011

(30) Foreign Application Priority Data
Oct. 24, 2008 (US) 61197190

(51) Int. Cl. A61K 31/7028; C07F 9/6561; A61P 33/06; A61K 31/675; A61P 31/18; C07D 493/04; A61K 31/635

(52) U.S. Cl. 514/25; 549/464; 549/220; 514/158; 514/81

(57) ABSTRACT

This invention relates to novel hydroxyethylamino sulfonamides and pharmaceutically acceptable salts thereof. This invention also provides compositions comprising a compound of this invention and the use of such compositions in methods of treating diseases and conditions that are beneficially treated by administering a compound with the ability to act as an HIV (human immunodeficiency virus) protease inhibitor.

### RELATED APPLICATION

This application claims the benefit of U.S. Provisional Application No. 61/197,190, filed Oct. 24, 2008. The entire teachings of the above application is incorporated herein by reference.

### BACKGROUND OF THE INVENTION

Many current medicines suffer from poor absorption, distribution, metabolism and/or excretion (ADME) properties that prevent their wider use. Poor ADME properties are also a major reason for the failure of drug candidates in clinical trials. While formulation technologies and prodrug strategies can be employed in some cases to improve certain ADME properties, these approaches fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment.

In some select cases, a metabolic inhibitor will be co-administered with a drug that is cleared too rapidly. Such is the case with the protease inhibitor class of drugs that are used to treat HIV infection. These drugs are typically co-dosed with ritonavir, an inhibitor of cytochrome P450 enzyme 3A4 (CYP3A4), the enzyme typically responsible for their metabolism. Ritonavir causes adverse effects and adds to the pill burden for HIV patients who must already take a combination of different drugs. Similarly, quinidine has been added to dextromethorphan for the purpose of reducing rapid CYP2D6 metabolism in a treatment of pseudobulbar affect. Quinidine, however, is a CYP2D6 inhibitor that has unwanted side effects that greatly limit its use in potential combination therapy.

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. This can cause those other drugs to accumulate in the body to toxic levels.

A potentially attractive strategy for improving a drug's metabolic properties is deuterium modification. In this approach, one attempts to slow the CYP-mediated metabolism of a drug by replacing one or more hydrogen atoms with deuterium atoms. Deuterium is a safe, stable, non-radioactive isotope of hydrogen. Deuterium forms stronger bonds with carbon than hydrogen does. In select cases, the increased bond strength imparted by deuterium can positively impact the ADME properties of a drug, creating the potential for improved drug efficacy, safety, and tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res, 1985, 14:1-40 (“Foster”); Kushner, D J et al, Can J Physiol Pharmacol, 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism. (See Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its undeuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

Darunavir, also known as Prezista™, or [(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b]furan-3-yl ester monoethanolate, selectively inhibits the cleavage of HIV encoded Gag-Pol polyproteins in infected cells, thereby preventing the formation of mature virus particles. (See FDA label for darunavir @ http://www.fda.gov/cder/foi/label/2006/021976s001lbl.pdf).

Darunavir is currently approved for treatment of HIV infection in combination with ritonavir and/or other antiretroviral agents.

The most common adverse events experienced by patients dosed with darunavir include, but are not limited to, diarrhea, nausea, abdominal pain, constipation, headache, common cold, increased amylase, neutropenia, and nasopharyngitis. Co-administration of darunavir is contraindicated with drugs that are highly dependent on CYP3A4 for clearance and for which elevated plasma concentrations are associated with serious and/or life-threatening events. (See FDA label for darunavir @ (http://www.fda.gov/cder/foi/label/2006/021976s001lbl.pdf).

Despite the beneficial activities of darunavir, there is a continuing need for new compounds to treat the aforementioned diseases and conditions.

### SUMMARY OF THE INVENTION

This invention relates to novel hydroxyethylamino sulfonamides, and pharmaceutically acceptable salts thereof. This invention also provides compositions comprising a compound of this invention and the use of such compositions in methods of treating diseases and conditions that are beneficially treated by administering a compound with the ability to act as an HIV (human immunodeficiency virus) protease inhibitor.

### DETAILED DESCRIPTION OF THE INVENTION

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of darunavir will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al, Seikagaku, 1994, 66:15; Gannes L Z et al., Comp Biochem Physiol Mol Integr Physiol, 1998, 119:725.

The term “isotopic enrichment factor” as used herein means the ratio between the isotopic abundance and the natural abundance of a specified isotope.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species that differs from a specific compound of this invention only in the isotopic composition thereof.

The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert”, “^{t}”, and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

The term “optionally substituted” refers to the optional replacement of one or more hydrogen atoms with another moiety. Unless otherwise specified, any hydrogen atom including a terminal hydrogen atom, can be optionally replaced.

The term “halo” refers to any of —Cl, —F, —Br, or —I.

The term “carboxy” refers to —C(O)OH

The term “oxo” refers to ═O.

The term “alkoxy” refers to —O-alkyl.

The term “alkylamino” refers to —NH-alkyl.

The term “dialkylamino” refers to N(alkyl)-alkyl, wherein the two alkyl moieties are the same or different.

The term “alkyl” refers to straight or branched alkyl chains of from 1 to 12 carbon atoms, unless otherwise specified. Examples of straight chained and branched alkyl groups include methyl, ethyl, n-propyl, iso-propyl, n-butyl, sec-butyl, tert-butyl, pentyl, hexyl, pentyl and octyl.

Examples of optional substituents on an alkyl group, such as a C₁₋₇ alkyl include halo, cyano, hydroxyl, carboxy, alkoxy, oxo, amino, alkylamino, dialkylamino, cycloheteroalkyl, aryl, and heteroaryl.

The term “cycloheteroalkyl” refers to an optionally substituted non-aromatic monocyclic, bicyclic, tricyclic, spirocyclic, or tetracyclic ring system which includes one or more heteroatoms such as nitrogen, oxygen or sulfur in at least one of the rings. Each ring can be four, five, six, seven or eight-membered. Examples include tetrahydrofuryl, tetrahyrothiophenyl, morpholino, thiomorpholino, pyrrolidinyl, piperazinyl, piperidinyl, and thiazolidinyl, along with the cyclic form of sugars. Suitable substituents on a cycloheteroalkyl can include, but are not limited to for example, alkyl, halo, cyano, hydroxyl, carboxy, alkoxy, oxo, amino, alkylamino and dialkylamino. Examples of alkyl substituted cycloheteroalkyls include, but are not limited to, 4-methylpiperazin-1-yl and 4-methylpiperidin-1-yl.

The term “aryl” refers to optionally substituted carbocyclic aromatic groups such as phenyl and naphthyl. Suitable substituents on an aryl can include, but are not limited to for example, alkyl, halo, cyano, hydroxyl, carboxy, alkoxy, amino, alkylamino and dialkylamino.

The term “heteroaryl” refers to an optionally substituted monocyclic aromatic group comprising one or more heteroatoms such as nitrogen, oxygen or sulfur in the ring, such as imidazolyl, thienyl, furyl, pyridyl, pyrimidyl, pyranyl, pyrazolyl, pyrrolyl, pyrazinyl, thiazolyl, oxazolyl, and tetrazolyl. Heteroaryl groups also include fused polycyclic aromatic ring systems in which at least one ring comprises one or more heteroatoms such as nitrogen, oxygen or sulfur. Examples include benzothienyl, benzofuryl, indolyl, quinolinyl, benzothiazole, benzoxazole, benzimidazole, quinolinyl, isoquinolinyl and isoindolyl. Suitable substituents on a heteroaryl can include, but are not limited to for example, alkyl, halo, cyano, hydroxyl, carboxy, alkoxy, amino, alkylamino and dialkylamino.

Unless otherwise specified, the term “α-amino acid” includes α-amino acids having a (D)-, (L)- or racemic (D,L) configuration. It is understood that when the variable R⁵ is an α-amino acid, it is linked to the rest of the molecule through the carbonyl carbon which is directly bonded to the α-carbon of the amino acid. In accordance with the structure of Formula I, such a linkage results in the formation of an ester.

### Therapeutic Compounds

The present invention provides a compound of Formula I:

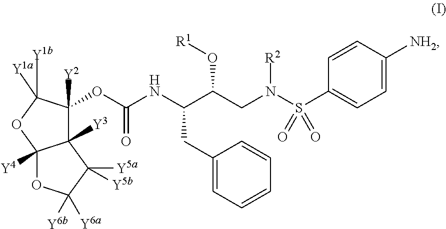

or a pharmaceutically acceptable salt thereof, wherein:
each Y is independently selected from hydrogen and deuterium;
at least one of Y⁴, Y^{5a}, Y^{5b}, Y^{6a} and Y^{6b} is deuterium;
R¹ is hydrogen or —(CR³R⁴—O)ₙ—R⁵;
R² is an isobutyl group having 0-9 deuterium;
R³ and R⁴ are independently selected from hydrogen and C₁-C₄ alkyl;
R⁵ is selected from an α-amino acid, —C(O)R⁶, —P(O)—(OM)₂ and —S(O)—OM;
R⁶ is hydrogen or an optionally substituted C₁-C₇ alkyl;
each M is hydrogen, or a cation independently selected from Li⁺, Na⁺, K⁺, Mg²⁺, Ca²⁺, Ba²⁺, and NH₄⁺; and
n is 0 or 1.

The term “isobutyl group having 0-9 deuterium” as used herein means a moiety of the formula —CX₂—CX—(CX₃)₂, where each X is independently selected from hydrogen and deuterium.

It will be readily apparent that when M is a divalent cation, such as Mg²⁺, Ca²⁺, or Ba²⁺, the ion will bind to a compound of Formula I in a molar ratio of 2 to 1 (compound of Formula I to M) when R⁵ is —S(O)—OM and in a molar ratio of 1:1 when R⁵ is —P(O)—(OM)₂.

One embodiment of this invention provides compounds of Formula I wherein R¹ is hydrogen or —(CH₂—O)ₙ—R⁵. In one aspect of this embodiment R⁵ is an α-amino acid with either the (D)-, (L)-, or racemic (D,L) configuration. In another aspect of this embodiment R⁵ is an α-amino acid having an (L)-configuration and selected from serine, lysine, tyrosine, valine, glutamic acid, aspartic acid, 3-pyridylalanine and histidine. In another aspect of this embodiment R⁵ is —C(O)R⁶, and R⁶ is a C₁-C₇ alkyl optionally substituted with halo, cyano, hydroxyl, carboxy, alkoxy, oxo, amino, alkylamino, dialkylamino, cycloheteroalkyl, aryl, arylalkyl, heteroaryl, or heteroarylalkyl, wherein any cyclic portion of the substituent is optionally further substituted. In yet another aspect R⁶ is selected from: —CH₂OCH₃; —CH₂CH₂OCH₃; —CH₂CH₂CO₂H; —CH₂CH₂NH₂; —CH₂CH₂NH—CH₃; —CH₂CH₂N(CH₃)₂;

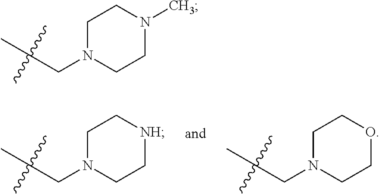

In an additional aspect, M is selected from Na⁺, Mg²⁺ and NH₄⁺.

In another embodiment of Formula I, R¹ is selected from —P(O)—(OM)₂ and —CH₂OP(O)—(OM)₂ and M is selected from Na⁺, Mg²⁺ and NH₄⁺. In a more specific embodiment M is Na⁺.

In an additional embodiment of Formula I, R¹ is hydrogen.

Another embodiment of this invention provides compounds of Formula I wherein R² is selected from —CH₂CH(CH₃)₂, —CH₂CH(CD₃)₂, —CH₂CD(CH₃)₂, —CD₂CH(CH₃)₂, —CH₂CD(CD₃)₂, —CD₂CH(CD₃)₂, —CD₂CD(CH₃)₂, and —CD₂CD(CD₃)₂. In a more specific embodiment R² is selected from —CH₂CH(CH₃)₂, —CH₂CD(CH₃)₂, —CD₂CH(CH₃)₂, —CH₂CD(CD₃)₂, —CD₂CD(CH₃)₂, and —CD₂CD(CD₃)₂. In an even more specific embodiment R² is selected from —CH₂CH(CH₃)₂, —CH₂CD(CD₃)₂ and —CD₂CD(CD₃)₂.

One embodiment of this invention provides compounds of Formula I wherein Y^{1a} and Y^{1b} are the same, Y^{5a} and Y^{5b} are the same; and Y^{6a} and Y^{6b} are the same. In one aspect of this embodiment, Y^{5a}, Y^{5b}, Y^{6a} and Y^{6b} are simultaneously deuterium.

In another embodiment of Formula I, R¹ is hydrogen; R² is selected from —CH₂CH(CH₃)₂, —CH₂CD(CD₃)₂; and —CD₂CD(CD₃)₂; each Y¹ is the same; each Y⁵ is the same; each Y⁶ is the same; and each Y is as defined in Table 1, below.

TABLE 1
Exemplary Compounds of the Invention

| Cmpd # | R² | each Y¹ | Y² | Y³ | Y⁴ | each Y⁵ | each Y⁶ |
|---|---|---|---|---|---|---|---|
| 100 | CH₂CH(CH₃)₂ | H | H | H | H | H | D |
| 101 | CH₂CH(CH₃)₂ | H | H | H | H | D | H |
| 102 | CH₂CH(CH₃)₂ | H | H | H | D | H | H |
| 103 | CH₂CH(CH₃)₂ | H | H | H | H | D | D |
| 104 | CH₂CH(CH₃)₂ | H | H | H | D | D | H |
| 105 | CH₂CH(CH₃)₂ | H | H | H | D | H | D |
| 106 | CH₂CH(CH₃)₂ | H | H | H | D | D | D |
| 107 | CH₂CH(CH₃)₂ | H | H | D | H | H | D |
| 108 | CH₂CH(CH₃)₂ | H | H | D | H | D | H |
| 109 | CH₂CH(CH₃)₂ | H | H | D | D | H | H |
| 110 | CH₂CH(CH₃)₂ | H | H | D | H | D | D |
| 111 | CH₂CH(CH₃)₂ | H | H | D | D | D | H |
| 112 | CH₂CH(CH₃)₂ | H | H | D | D | H | D |
| 113 | CH₂CH(CH₃)₂ | H | H | D | D | D | D |
| 114 | CH₂CH(CH₃)₂ | H | D | H | H | H | D |
| 115 | CH₂CH(CH₃)₂ | H | D | H | H | D | H |
| 116 | CH₂CH(CH₃)₂ | H | D | H | D | H | H |
| 117 | CH₂CH(CH₃)₂ | H | D | H | H | D | D |
| 118 | CH₂CH(CH₃)₂ | H | D | H | D | D | H |
| 119 | CH₂CH(CH₃)₂ | H | D | H | D | H | D |
| 120 | CH₂CH(CH₃)₂ | H | D | H | D | D | D |
| 121 | CH₂CH(CH₃)₂ | D | H | H | H | H | D |
| 122 | CH₂CH(CH₃)₂ | D | H | H | H | D | H |
| 123 | CH₂CH(CH₃)₂ | D | H | H | D | H | H |
| 124 | CH₂CH(CH₃)₂ | D | H | H | H | D | D |
| 125 | CH₂CH(CH₃)₂ | D | H | H | D | D | H |
| 126 | CH₂CH(CH₃)₂ | D | H | H | D | H | D |
| 127 | CH₂CH(CH₃)₂ | D | H | H | D | D | D |
| 128 | CH₂CH(CH₃)₂ | H | D | D | H | H | D |
| 129 | CH₂CH(CH₃)₂ | H | D | D | H | D | H |
| 130 | CH₂CH(CH₃)₂ | H | D | D | D | H | H |
| 131 | CH₂CH(CH₃)₂ | H | D | D | H | D | D |
| 132 | CH₂CH(CH₃)₂ | H | D | D | D | D | H |
| 133 | CH₂CH(CH₃)₂ | H | D | D | D | H | D |
| 134 | CH₂CH(CH₃)₂ | H | D | D | D | D | D |
| 135 | CH₂CH(CH₃)₂ | D | D | H | H | H | D |
| 136 | CH₂CH(CH₃)₂ | D | D | H | H | D | H |
| 137 | CH₂CH(CH₃)₂ | D | D | H | D | H | H |
| 138 | CH₂CH(CH₃)₂ | D | D | H | H | D | D |
| 139 | CH₂CH(CH₃)₂ | D | D | H | D | D | H |
| 140 | CH₂CH(CH₃)₂ | D | D | H | D | H | D |
| 141 | CH₂CH(CH₃)₂ | D | D | H | D | D | D |
| 142 | CH₂CH(CH₃)₂ | D | H | D | H | H | D |
| 143 | CH₂CH(CH₃)₂ | D | H | D | H | D | H |
| 144 | CH₂CH(CH₃)₂ | D | H | D | D | H | H |
| 145 | CH₂CH(CH₃)₂ | D | H | D | H | D | D |
| 146 | CH₂CH(CH₃)₂ | D | H | D | D | D | H |
| 147 | CH₂CH(CH₃)₂ | D | H | D | D | H | D |
| 148 | CH₂CH(CH₃)₂ | D | H | D | D | D | D |
| 149 | CH₂CH(CH₃)₂ | D | D | D | H | H | D |
| 150 | CH₂CH(CH₃)₂ | D | D | D | H | D | H |
| 151 | CH₂CH(CH₃)₂ | D | D | D | D | H | H |
| 152 | CH₂CH(CH₃)₂ | D | D | D | H | D | D |
| 153 | CH₂CH(CH₃)₂ | D | D | D | D | D | H |
| 154 | CH₂CH(CH₃)₂ | D | D | D | D | H | D |
| 155 | CH₂CH(CH₃)₂ | D | D | D | D | D | D |
| 156 | CH₂CD(CD₃)₂ | H | H | H | H | H | D |
| 157 | CH₂CD(CD₃)₂ | H | H | H | H | D | H |
| 158 | CH₂CD(CD₃)₂ | H | H | H | D | H | H |
| 159 | CH₂CD(CD₃)₂ | H | H | H | H | D | D |
| 160 | CH₂CD(CD₃)₂ | H | H | H | D | D | H |
| 161 | CH₂CD(CD₃)₂ | H | H | H | D | H | D |
| 162 | CH₂CD(CD₃)₂ | H | H | H | D | D | D |
| 163 | CH₂CD(CD₃)₂ | H | H | D | H | H | D |
| 164 | CH₂CD(CD₃)₂ | H | H | D | H | D | H |
| 165 | CH₂CD(CD₃)₂ | H | H | D | D | H | H |
| 166 | CH₂CD(CD₃)₂ | H | H | D | H | D | D |
| 167 | CH₂CD(CD₃)₂ | H | H | D | D | D | H |
| 168 | CH₂CD(CD₃)₂ | H | H | D | D | H | D |
| 169 | CH₂CD(CD₃)₂ | H | H | D | D | D | D |
| 170 | CH₂CD(CD₃)₂ | H | D | H | H | H | D |
| 171 | CH₂CD(CD₃)₂ | H | D | H | H | D | H |
| 172 | CH₂CD(CD₃)₂ | H | D | H | D | H | H |
| 173 | CH₂CD(CD₃)₂ | H | D | H | H | D | D |
| 174 | CH₂CD(CD₃)₂ | H | D | H | D | D | H |
| 175 | CH₂CD(CD₃)₂ | H | D | H | D | H | D |
| 176 | CH₂CD(CD₃)₂ | H | D | H | D | D | D |
| 177 | CH₂CD(CD₃)₂ | D | H | H | H | H | D |
| 178 | CH₂CD(CD₃)₂ | D | H | H | H | D | H |
| 179 | CH₂CD(CD₃)₂ | D | H | H | D | H | H |
| 180 | CH₂CD(CD₃)₂ | D | H | H | H | D | D |
| 181 | CH₂CD(CD₃)₂ | D | H | H | D | D | H |
| 182 | CH₂CD(CD₃)₂ | D | H | H | D | H | D |
| 183 | CH₂CD(CD₃)₂ | D | H | H | D | D | D |
| 184 | CH₂CD(CD₃)₂ | H | D | D | H | H | D |
| 185 | CH₂CD(CD₃)₂ | H | D | D | H | D | H |
| 186 | CH₂CD(CD₃)₂ | H | D | D | D | H | H |
| 187 | CH₂CD(CD₃)₂ | H | D | D | H | D | D |
| 188 | CH₂CD(CD₃)₂ | H | D | D | D | D | H |
| 189 | CH₂CD(CD₃)₂ | H | D | D | D | H | D |
| 190 | CH₂CD(CD₃)₂ | H | D | D | D | D | D |
| 191 | CH₂CD(CD₃)₂ | D | D | H | H | H | D |
| 192 | CH₂CD(CD₃)₂ | D | D | H | H | D | H |
| 193 | CH₂CD(CD₃)₂ | D | D | H | D | H | H |
| 194 | CH₂CD(CD₃)₂ | D | D | H | H | D | D |
| 195 | CH₂CD(CD₃)₂ | D | D | H | D | D | H |
| 196 | CH₂CD(CD₃)₂ | D | D | H | D | H | D |
| 197 | CH₂CD(CD₃)₂ | D | D | H | D | D | D |
| 198 | CH₂CD(CD₃)₂ | D | H | D | H | H | D |
| 199 | CH₂CD(CD₃)₂ | D | H | D | H | D | H |
| 200 | CH₂CD(CD₃)₂ | D | H | D | D | H | H |
| 201 | CH₂CD(CD₃)₂ | D | H | D | H | D | D |
| 202 | CH₂CD(CD₃)₂ | D | H | D | D | D | H |
| 203 | CH₂CD(CD₃)₂ | D | H | D | D | H | D |
| 204 | CH₂CD(CD₃)₂ | D | H | D | D | D | D |
| 205 | CH₂CD(CD₃)₂ | D | D | D | H | H | D |
| 206 | CH₂CD(CD₃)₂ | D | D | D | H | D | H |
| 207 | CH₂CD(CD₃)₂ | D | D | D | D | H | H |
| 208 | CH₂CD(CD₃)₂ | D | D | D | H | D | D |
| 209 | CH₂CD(CD₃)₂ | D | D | D | D | D | H |
| 210 | CH₂CD(CD₃)₂ | D | D | D | D | H | D |
| 211 | CH₂CD(CD₃)₂ | D | D | D | D | D | D |
| 212 | CD₂CD(CD₃)₂ | H | H | H | H | H | D |
| 213 | CD₂CD(CD₃)₂ | H | H | H | H | D | H |
| 214 | CD₂CD(CD₃)₂ | H | H | H | D | H | H |
| 215 | CD₂CD(CD₃)₂ | H | H | H | H | D | D |
| 216 | CD₂CD(CD₃)₂ | H | H | H | D | D | H |
| 217 | CD₂CD(CD₃)₂ | H | H | H | D | H | D |
| 218 | CD₂CD(CD₃)₂ | H | H | H | D | D | D |
| 219 | CD₂CD(CD₃)₂ | H | H | D | H | H | D |
| 220 | CD₂CD(CD₃)₂ | H | H | D | H | D | H |
| 221 | CD₂CD(CD₃)₂ | H | H | D | D | H | H |
| 222 | CD₂CD(CD₃)₂ | H | H | D | H | D | D |
| 223 | CD₂CD(CD₃)₂ | H | H | D | D | D | H |
| 224 | CD₂CD(CD₃)₂ | H | H | D | D | H | D |
| 225 | CD₂CD(CD₃)₂ | H | H | D | D | D | D |
| 226 | CD₂CD(CD₃)₂ | H | D | H | H | H | D |
| 227 | CD₂CD(CD₃)₂ | H | D | H | H | D | H |
| 228 | CD₂CD(CD₃)₂ | H | D | H | D | H | H |
| 229 | CD₂CD(CD₃)₂ | H | D | H | H | D | D |
| 230 | CD₂CD(CD₃)₂ | H | D | H | D | D | H |
| 231 | CD₂CD(CD₃)₂ | H | D | H | D | H | D |
| 232 | CD₂CD(CD₃)₂ | H | D | H | D | D | D |
| 233 | CD₂CD(CD₃)₂ | D | H | H | H | H | D |
| 234 | CD₂CD(CD₃)₂ | D | H | H | H | D | H |
| 235 | CD₂CD(CD₃)₂ | D | H | H | D | H | H |
| 236 | CD₂CD(CD₃)₂ | D | H | H | H | D | D |
| 237 | CD₂CD(CD₃)₂ | D | H | H | D | D | H |
| 238 | CD₂CD(CD₃)₂ | D | H | H | D | H | D |
| 239 | CD₂CD(CD₃)₂ | D | H | H | D | D | D |
| 240 | CD₂CD(CD₃)₂ | H | D | D | H | H | D |
| 241 | CD₂CD(CD₃)₂ | H | D | D | H | D | H |
| 242 | CD₂CD(CD₃)₂ | H | D | D | D | H | H |
| 243 | CD₂CD(CD₃)₂ | H | D | D | H | D | D |
| 244 | CD₂CD(CD₃)₂ | H | D | D | D | D | H |
| 245 | CD₂CD(CD₃)₂ | H | D | D | D | H | D |
| 246 | CD₂CD(CD₃)₂ | H | D | D | D | D | D |
| 247 | CD₂CD(CD₃)₂ | D | D | H | H | H | D |
| 248 | CD₂CD(CD₃)₂ | D | D | H | H | D | H |
| 249 | CD₂CD(CD₃)₂ | D | D | H | D | H | H |
| 250 | CD₂CD(CD₃)₂ | D | D | H | H | D | D |
| 251 | CD₂CD(CD₃)₂ | D | D | H | D | D | H |
| 252 | CD₂CD(CD₃)₂ | D | D | H | D | H | D |
| 253 | CD₂CD(CD₃)₂ | D | D | H | D | D | D |
| 254 | CD₂CD(CD₃)₂ | D | H | D | H | H | D |
| 255 | CD₂CD(CD₃)₂ | D | H | D | H | D | H |
| 256 | CD₂CD(CD₃)₂ | D | H | D | D | H | H |
| 257 | CD₂CD(CD₃)₂ | D | H | D | H | D | D |
| 258 | CD₂CD(CD₃)₂ | D | H | D | D | D | H |
| 259 | CD₂CD(CD₃)₂ | D | H | D | D | H | D |
| 260 | CD₂CD(CD₃)₂ | D | H | D | D | D | D |
| 261 | CD₂CD(CD₃)₂ | D | D | D | H | H | D |
| 262 | CD₂CD(CD₃)₂ | D | D | D | H | D | H |
| 263 | CD₂CD(CD₃)₂ | D | D | D | D | H | H |
| 264 | CD₂CD(CD₃)₂ | D | D | D | H | D | D |
| 265 | CD₂CD(CD₃)₂ | D | D | D | D | D | H |
| 266 | CD₂CD(CD₃)₂ | D | D | D | D | H | D |
| and 267 | CD₂CD(CD₃)₂ | D | D | D | D | D | D |

or a pharmaceutically acceptable salt of any of the foregoing.

In one embodiment, the compound of Formula I is a compound of the Formula Ia:

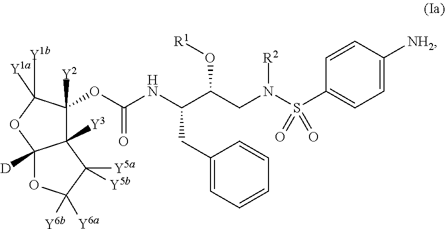

or a pharmaceutically acceptable salt thereof, wherein each Y is independently selected from hydrogen and deuterium.

In one embodiment, the compound of Formula I is a compound of the Formula Ib:

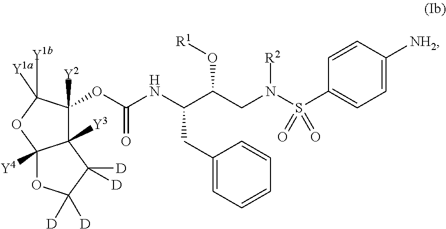

or a pharmaceutically acceptable salt thereof, wherein each Y is independently selected from hydrogen and deuterium.

Examples of specific compounds of this invention include the following:

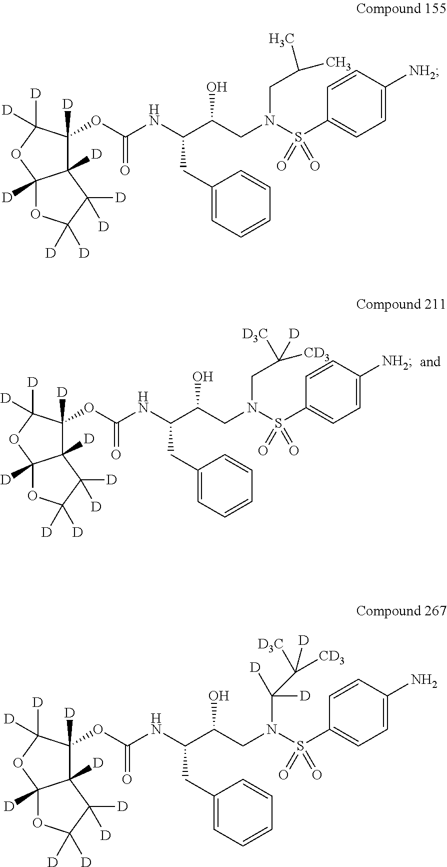

or a pharmaceutically acceptable salt thereof.

Further examples of specific compounds of this invention include the following:

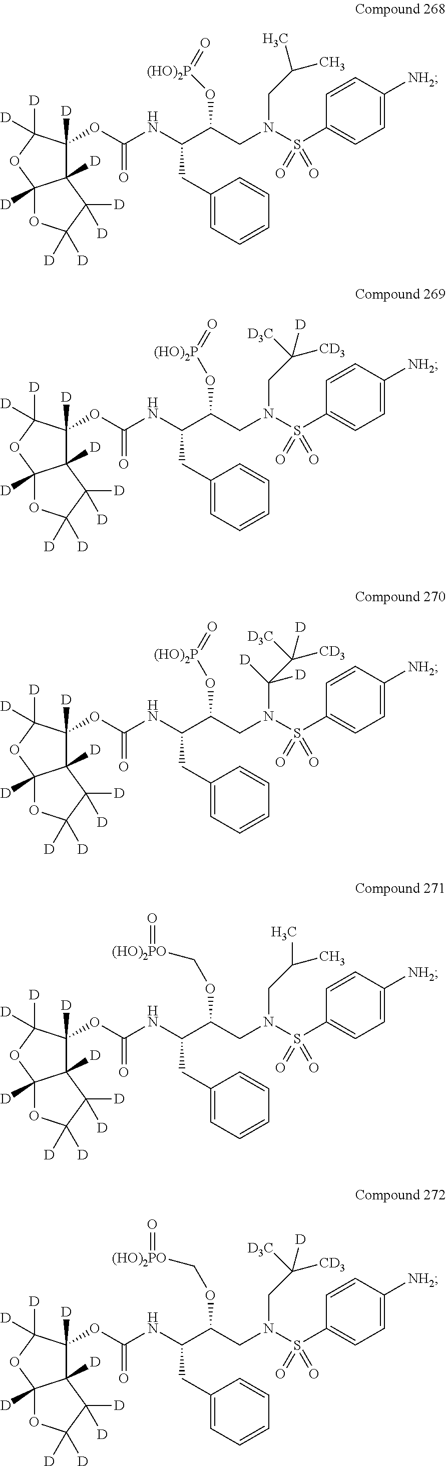

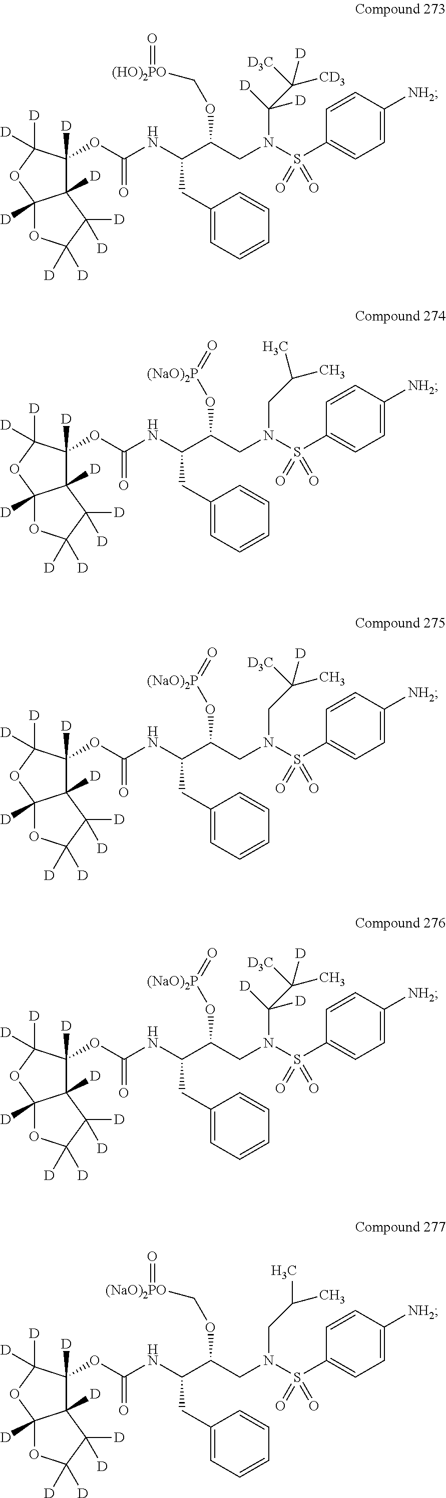

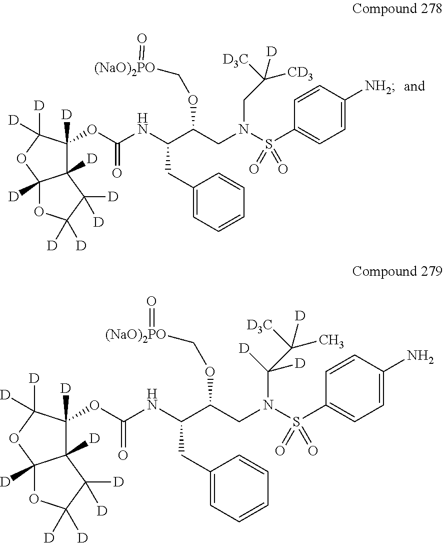

or pharmaceutically acceptable salt thereof.

The present invention provides additionally for novel intermediates of Formula IIa:

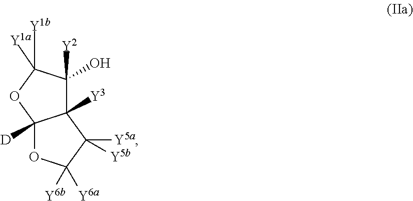

or a salt thereof wherein:

each Y is independently selected from hydrogen and deuterium.

Examples of intermediates of Formula IIa include:

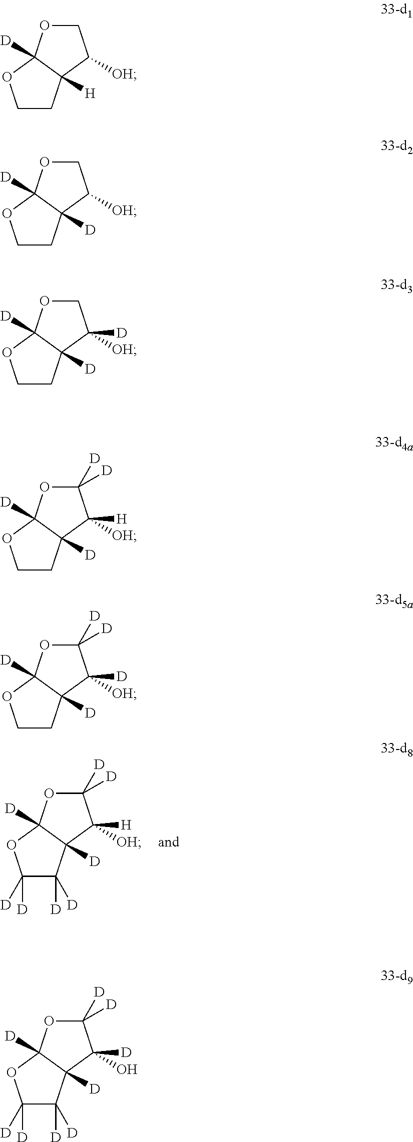

or a salt thereof.

The present invention also provides for novel intermediates of Formula IIb:

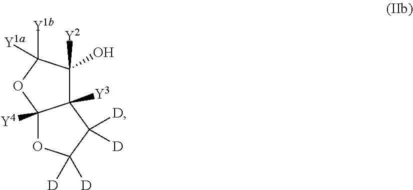

or a salt thereof
wherein: each Y is independently selected from hydrogen and deuterium.

Examples intermediates of Formula IIb include:

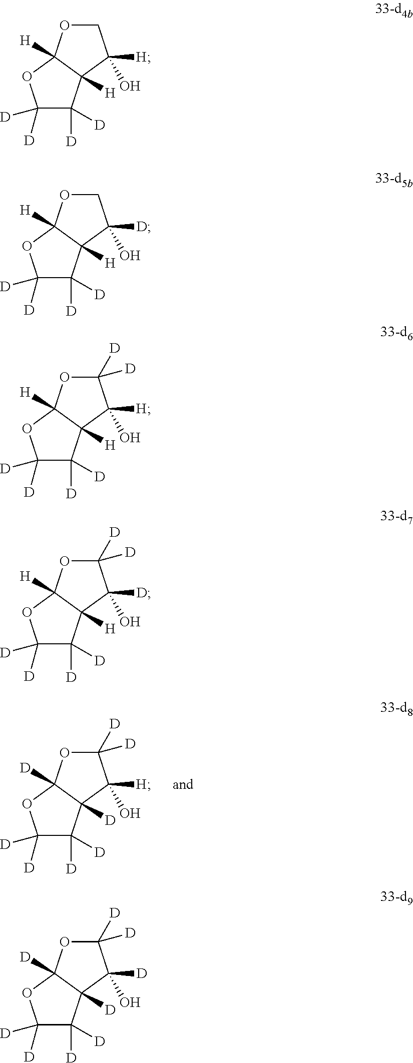

or a salt thereof.

In another set of embodiments, any atom not designated as deuterium in any of the embodiments set forth above is present at its natural isotopic abundance.

The synthesis of compounds of Formula I can be readily achieved by synthetic chemists of ordinary skill by reference to the Exemplary Synthesis and Examples disclosed herein. Relevant procedures and intermediates are disclosed, for instance in Ghosh, A K et al., J Org Chem, 2004, 69: 7822-7829; Ghosh, A K et al., J Med Chem, 2005, 48: 1813-1822; Ghosh, A K et al., J Med Chem, 2006, 49: 5252-5261; Doan, B D et al., US Patent App Pub No US 2005/0261507.

Such methods for making darunavir can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of Formula I is depicted in Schemes 1-4, below.

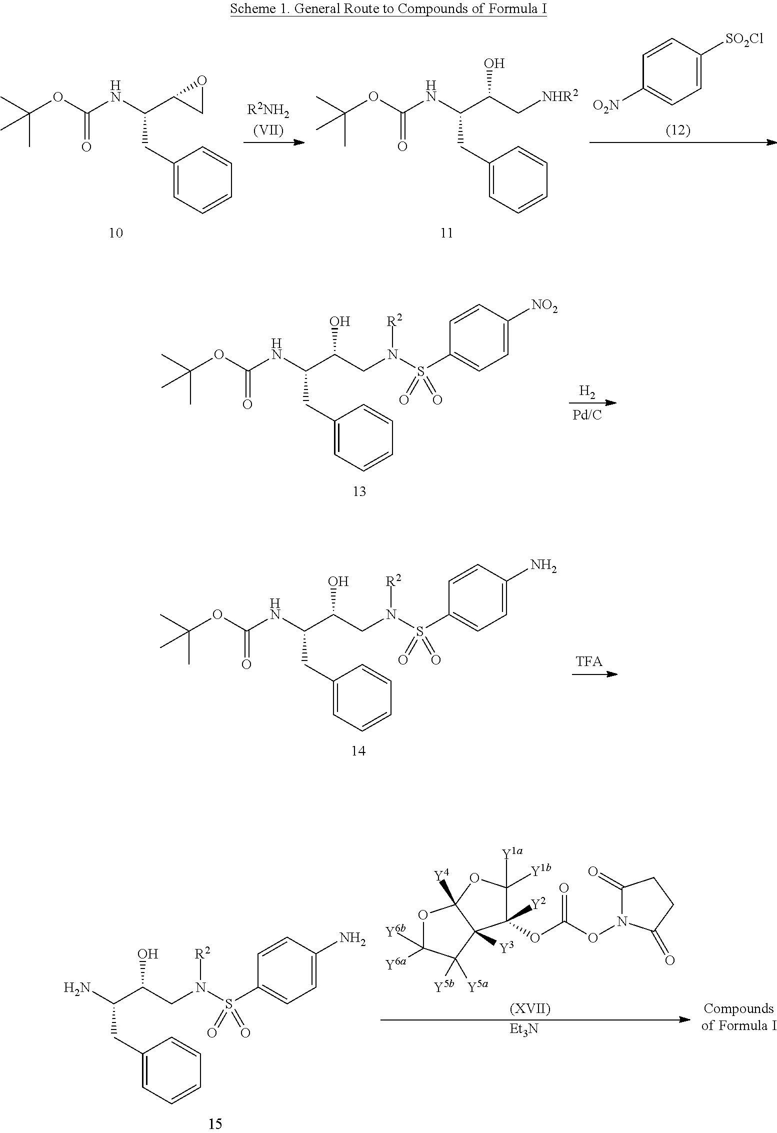

Scheme 1 above shows a general route to prepare compounds of Formula I. Commercially available enantiopure epoxide 10 is opened with the substituted isobutyl amine VII in hot isopropanol to provide the secondary amine 11. This amine is then reacted with sulfonyl chloride 12 and NaHCO₃ in dichloromethane to provide the sulfonamide 13, which is then reduced to the aniline 14 by hydrogenation over palladium on carbon. Trifluoroacetic acid treatment, or alternatively hydrochloric acid treatment, to remove the BOC group provides 15, which is then coupled with the mixed carbonate XVII in the presence of Et₃N to provide compounds of Formula I.

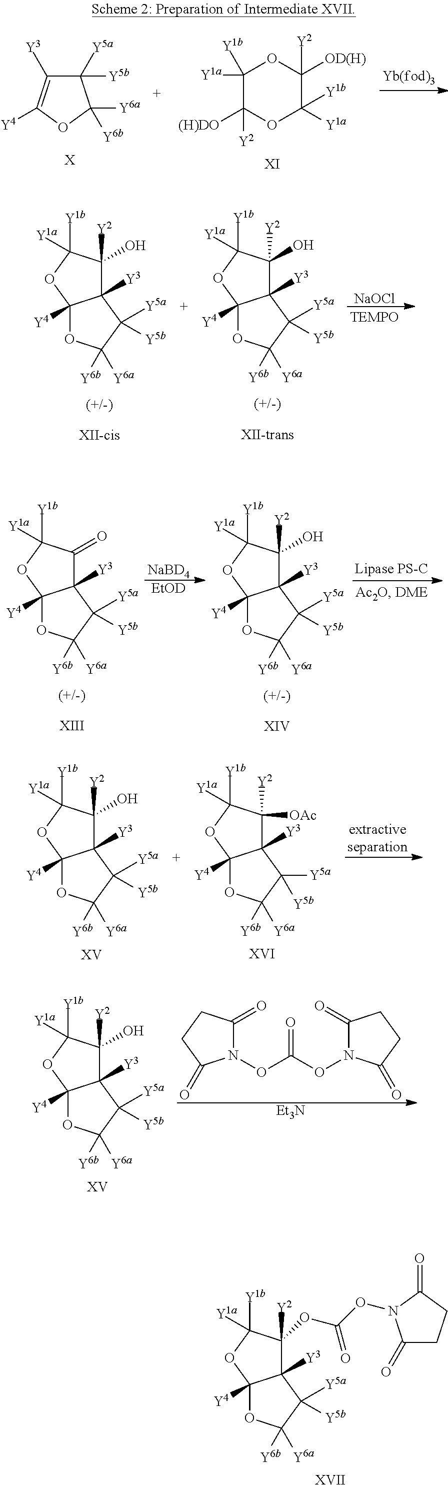

The appropriately deuterated analogs of intermediate XVII can be prepared according to the procedures disclosed by Yu, R. H. et al., in Organic Process Research and Development 2007, 11: 972 using the appropriately deuterated materials as shown in Scheme 2. The appropriately deuterated dihydrofuran X (the various deuterated forms of X can be prepared from succinic anhydride, from dihydrofuran, and from γ-butyrolactone as described in Keay, B A et al., JOC, 2007, 72: 7252-7259) is reacted with the deuterated glycolaldehyde dimer XI (the glycolaldehyde XI, where each Y is deuterium, can be prepared from dihydroxyfumaric acid or dihydroxymaleic acid by thermal

decarboxylation in D₂O as described in Wong, C-H and Whitesides, G M, JACS, 1983, 105: 5012-5014) in the presence of catalytic ytterbium tris(6,6,7,7,8,8,8-heptafluoro-2,2-dimethyl-3,5-octanedionate “Yb(fod)₃” to afford a diastereomeric mixture of the bicyclic alchohols XII. Subsequent oxidation of XII with sodium hypochlorite and 2,2,6,6-tetramethylpiperidine-1-oxyl “TEMPO” gives the corresponding ketone XIII which is reduced with sodium borodeuteride in d1-ethanol to afford the racemic cis-bisfuran alcohol XIV. Resolution of the racemic alcohol XIV with lipase PS-C amano I enzyme gives the resolved enantiomer of cis-bisfuran alcohol XV and the antipode cis-bisfuran acetate XVI. Via an extractive workup the undesired antipode acetate is extracted away into the organic layer, allowing the desired alcohol XV to be isolated from the aqueous layer. Appropriately deuterated analogs of XV can then be converted to the mixed carbonate XVII by reaction with disuccinimidyl carbonate and triethylamine in acetonitrile as described by Ghosh, A K et al., in J Org Chem, 2004, 69: 7822-7829.

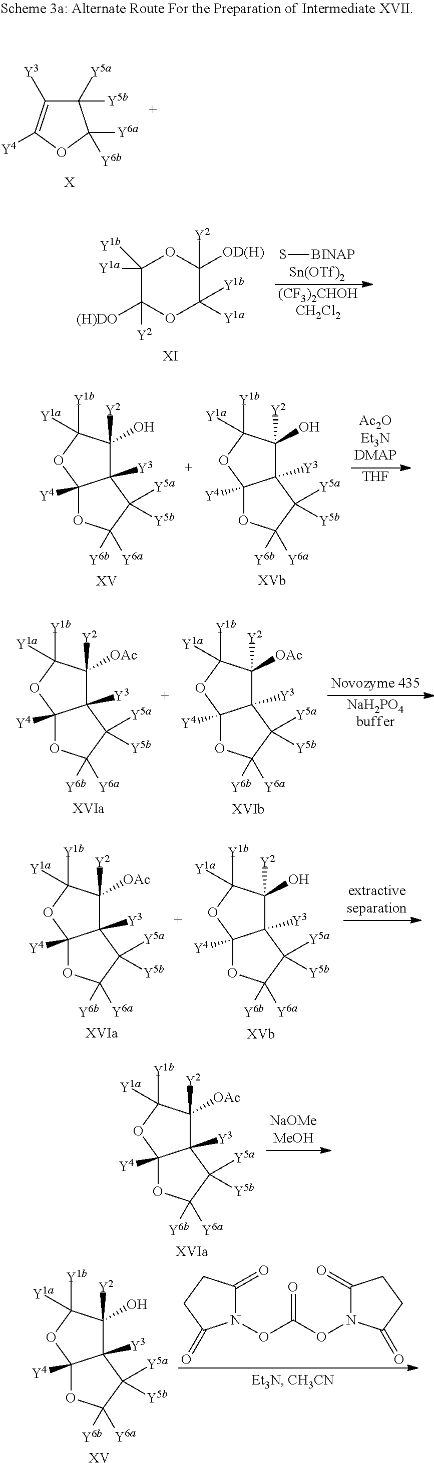

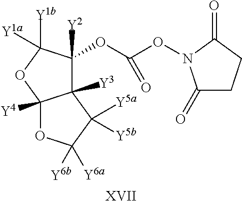

An alternative approach to the preparation of intermediate XVII utilizes conditions described in Canoy, W L et al., Org Lett, 2008, 10(6):1103-1106 to form the enantiopure cis-bisfuran alcohol XV as shown in Scheme 3a above. The appropriately deuterated dihydrofuran X (see description for Scheme 2 above) is coupled with the appropriately deuterated glycolaldehyde dimer XI (see description for Scheme 2 above) in the presence of S-BINAP and tin triflate to yield an enantiomeric mixture of XV and XVb. Acylation of these cis-bisfurans using acetic anhydride affords the enantiomeric mixture of XVIa and XVIb. Selective hydrolysis of XVIb to XVb using the lipase Novozyme 435, CAS No. 9001-62-1 is followed by organic extraction to isolate the desired acylated cis-bisfuran XVIa. Hydrolysis of XVIa in base affords the enantiopure cis-bisfuran alcohol XV which is reacted with disuccinimidyl carbonate as described above (Ghosh, et al.) to yield intermediate XVII. If Y² in the enantiopure cis-bisfuran alcohol XV is hydrogen, it can be converted to deuterium by oxidation with a Dess-Martin reagent in CH₂Cl₂, followed by reduction with sodium borodeuteride in deuterated ethanol prior to reaction with disuccinimidyl carbonate.

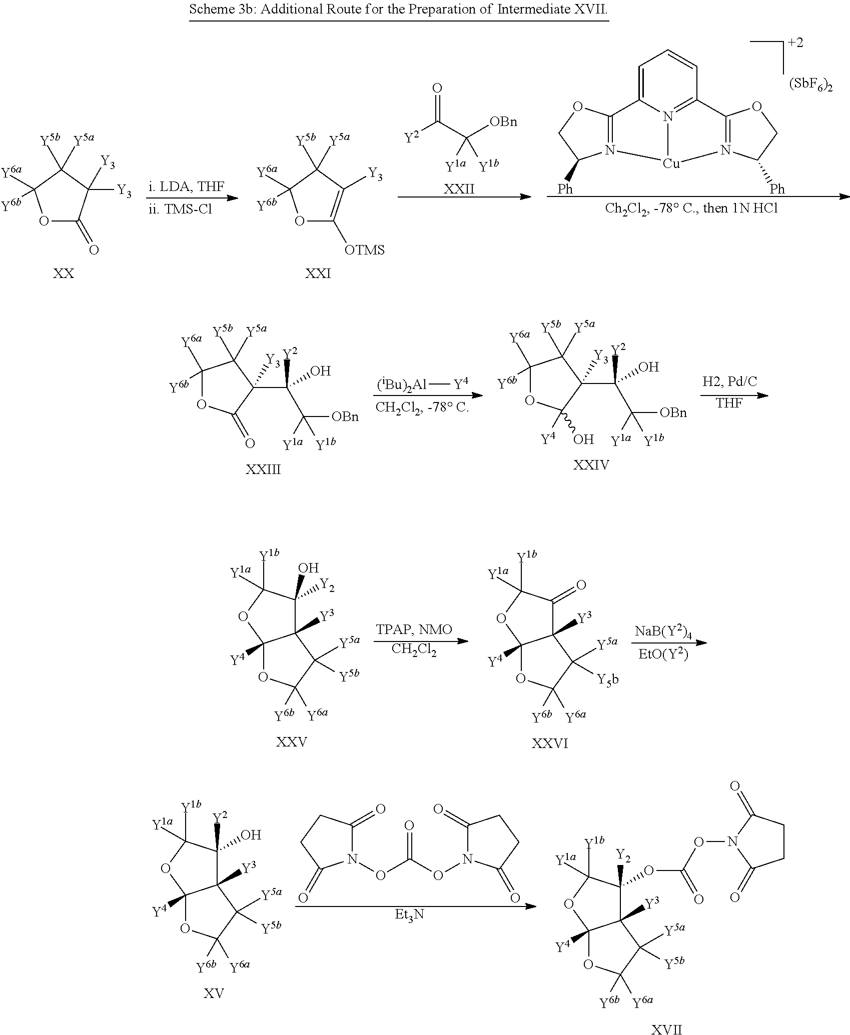

Another alternative approach to the preparation of intermediate XVII is shown in Scheme 3b using methods analagous to those of Xie, S et al, Tetrahedron Asymmetry 2008, 19:2015. An appropriately deuterated gamma-butyrolactone (XX) is treated with lithium diisopropylamide to generate the corresponding lithium enolate which is subsequently trapped with trimethylsilyl-chloride (TMS-Cl) to afford the trimethylsilyl ketene acetal XXI. The Mukaiyama aldol reaction of XXI with an appropriately deuterated benzyloxyacetaldehyde (XXII) in the presence of a chiral copper catalyst (prepared according to the procedure of Evans, D A et al, J Am Chem Soc 1996, 118: 5814) gives alcohol XXIII as the major diastereomer. Reduction of the lactone with diisobutylaluminum hydride (or diisobutylaluminum deuteride) affords a diastereomeric mixture of lactols (XXIV). Atmospheric hydrogenolysis with a palladium catalyst in THF forms the primary alcohol which cyclizes spontaneously to form the bis-THF alcohol XXV. Oxidation of the alcohol is carried out with 4-methylmorpholine-N-oxide (NMO) in the presence of catalytic tetrapropylammonium perruthenate (TPAP) to give ketone XXVI. Reduction of the bicyclic ketone XXVI with sodium borohydride or sodium borodeuteride affords the appropriately deuterated chiral cis-bisfuran alcohol XV. Finally, reaction of XV with disuccinimidyl carbonate and triethylamine in acetonitrile in a manner analogous to Ghosh, A K et al, J Org Chem 2004, 69:7822 affords the desired appropriately deuterated mixed carbonate XVII.

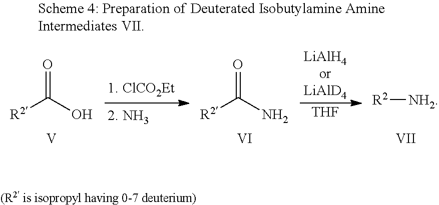

The deuterated analogs of isobutylamine VII can be prepared as shown in Scheme 4. Deuterated isobutyric acid V is activated as the mixed anhydride with ethyl chloroformate and then reacted with ammonia to provide the amide VI according to the general procedure for amide formation disclosed by Alvarado, C et al., Tet Lett, 2007, 48: 603-607. Alternatively, carbonyldiimidazole may be used in place of ethyl chloroformate. The isobutyric acid amide VI can be readily converted to the isobutyl amine by reduction with lithium aluminum hydride or lithium aluminum deuteride in a manner analogous to the procedures disclosed by Poehler, T et al., Eur J Med Chem, 2007, 42: 175-197.

The following deuterated isobutyric acids are commercially available and may be used in Scheme 4:

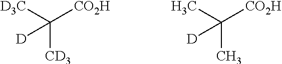

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free pharmaceutical compositions comprising an effective amount of a compound of Formula I (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament. Throughout the application, all references to compounds of Formula I or compounds of the invention include pharmaceutically acceptable salts of such compounds unless specifically stated otherwise.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as darunavir. Such agents include those indicated as being useful in combination with darunavir, including but not limited to, those described in WO 2003049746, WO 2005027855, and WO 2006005720.

Preferably, the second therapeutic agent is an agent useful in the treatment or prevention of a disease including, but not limited to, (HIV) infection and malaria.

In one embodiment, the second therapeutic agent is selected from other anti-retroviral agents including, but not limited to, a second HIV protease inhibitor (e.g., amprenavir, fosamprenavir, tipranavir, indinavir, saquinavir, lopinavir, ritonavir, darunavir, or nelfinavir), a non-nucleoside reverse transcriptase inhibitor (“NNRTI”) (e.g., etravirine, delavirdine, efavirenz, nevirapine, or rilpivirine), a nucleoside/nucleotide reverse transcriptase inhibitor (“NRTI”) (e.g., zidovudine, lamivudine, emtricitabine, tenofovir disoproxil fumarate, didanosine, stavudine, abacavir, racivir, amdoxovir, apricitabine, entecavir, adefovir or elvucitabine) a viral entry inhibitor (e.g., enfuvirtide, maraviroc, vicriviroc, PRO 140, or TNX-355), an integrase inhibitor (e.g., raltegravir, or elvitegravir), an immune based antiretroviral agent (e.g., immunitin, proleukin, remune, BAY 50-4798 or IR103), a viral maturation inhibitor (e.g., bevirimat), a cellular inhibitor (e.g., droxia or hydroxyurea), or combinations of two or more of the above.

In one embodiment, the second therapeutic agent is selected from ritonavir, atazanavir, indinavir, TMC125 (etravirine), tenofovir, emtricitabine, zidovudine, lopinavir, efavirenz, fosamprenavir, tipranavir, nevirapine, lamivudine, abacavir and combinations thereof. (See label for darunavir at http://www.fda.gov/cder/foi/label/2006/021976s001lbl.pdf and see clinical trials using darunavir at http://clinicaltrials.gov/ct/search?term=darunavir.)

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., Cancer Chemother. Rep, 1966, 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 1 mg to about 6000 mg per treatment. In more specific embodiments the range is from about 10 mg to 3000 mg, or from about 20 mg to 1200 mg, or most specifically from about 100 mg to 600 mg per treatment. Treatment typically is administered twice daily. In one embodiment, a compound of this invention is administered without co-administration of ritonavir.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for darunavir.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of inhibiting the activity of HIV protease in an infected cell, comprising contacting such cell with one or more compounds of Formula I herein.

According to another embodiment, the invention provides a method of treating a disease that is beneficially treated by darunavir in a patient in need thereof comprising the step of administering to said patient an effective amount of a compound or pharmaceutically acceptable salt thereof or a composition of this invention. Such diseases are well known in the art and are disclosed in, but not limited to the following patents and published applications: WO 1994004492, WO 1995006030, U.S. Pat. No. 6,335,460, and WO 2005027855. Such diseases include, but are not limited to, human immunodeficiency virus (HIV) infection and malaria.

In one particular embodiment, the method of this invention is used to treat HIV infection in a patient in need thereof.

Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with darunavir. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula I and a second therapeutic agent for the treatment of HIV infection, wherein the second therapeutic agent is selected from one or more of ritonavir, atazanavir, indinavir, etravirine, tenofovir, emtricitabine, zidovudine, lopinavir, efavirenz, fosamprenavir, tipranavir, nevirapine, lamivudine, and abacavir. (See clinical trials including darunavir @ http://clinicaltrials.gov).

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

In one aspect, the compound of Formula I or a composition comprising a compound of Formula I is for use in treating an HIV infection.

In another aspect, the compound of Formula I or a composition comprising a compound of Formula I is for use in treating an HIV infection; and the compound or composition is used in conjunction with one or more of ritonavir, atazanavir, indinavir, etravirine, tenofovir, emtricitabine, zidovudine, lopinavir, efavirenz, fosamprenavir, tipranavir, nevirapine, lamivudine, and abacavir.

In one aspect, the compound of Formula I or a composition comprising a compound of Formula I is for use in treating an HIV infection; and the compound or composition is not used in conjunction with ritonavir.

The term “used in conjunction with” as used herein means administered simultaneously with, or administered within 24 hours of the subject compound(s).

### Pharmaceutical Kits

The present invention also provides kits for use to treat HIV infection. These kits comprise (a) a pharmaceutical composition comprising a compound of Formula I or a salt thereof, wherein said pharmaceutical composition is in a container; and (b) instructions describing a method of using the pharmaceutical composition to treat HIV infection.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### EXAMPLES

### Example 1

### Synthesis of 4-Amino-N-((2R,3S)-3-amino-2-hydroxy-4-phenylbutyl)-N-(isobutyl-d₉)-benzenesulfonamide (24-d₉)

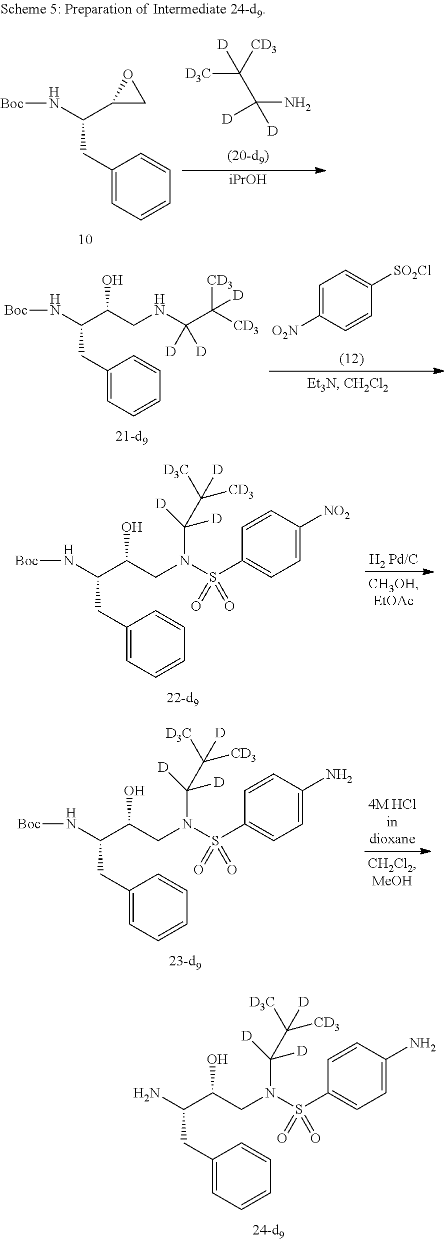

Step 1. tert-Butyl (2S,3R)-3-Hydroxy-4-((isobutyl-d₉)-amino)-1-phenylbutan-2-ylcarbamate (21-d₉). A mixture of commercially-available tert-butyl (S)-1-((S)-oxiran-2-yl)-2-phenylethyl-carbamate (10) (1.0 g, 3.8 mmol) and 2-(methylpropyl-d₉)-amine (20-d₉) (0.5 g, 6.08 mmol, CDN Isotopes, 98 atom % D) in isopropanol (30 mL) was stirred at reflux under nitrogen for 6 hours. The reaction mixture was allowed to cool overnight. The solvent was removed under reduced pressure to give crude 21-d₉ that was used directly in the next step without further purification.

Step 2. tert-Butyl (2S,3R)-3-hydroxy-4-(N-(isobutyl-d₉)-4-nitrophenylsulfonamido)-1-phenylbutan-2-ylcarbamate (22-d₉). To a solution of crude 21-d₉ (assumed 3.8 mmol) in dichloromethane (25 mL) was added triethylamine (0.46 g, 4.56 mmol, 1.2 equiv) followed by the addition of a solution of 4-nitrobenzenesulfonyl chloride (12) (0.84 g, 3.8 mmol, 1 equiv) in dichloromethane (5 mL). The reaction mixture was stirred overnight at room temperature. The mixture was diluted with dichloromethane (100 mL) and washed with water (2×60 mL), brine (60 mL), dried over sodium sulfate and filtered. The solvent was removed under reduced pressure and the crude product was purified by chromatography on silica gel (60 g), eluting with 1% ethyl acetate in dichloromethane (3 L) to give 1.28 g (64% over 2 steps) of 22-d₉.

Step 3. tert-Butyl (2S,3R)-4-(4-Amino-N-(isobutyl-d₉)-phenylsulfonamido)-3-hydroxy-1-phenylbutan-2-ylcarbamate (23-d₉). To a solution of 22-d₉ (1.26 g, 2.37 mmol) in methanol (30 mL) and ethyl acetate (30 mL) was added 20% palladium on activated carbon (50% wet, 0.20 g). The mixture was subjected to hydrogenation at 40 psi for 2.5 hours. The mixture was filtered through a pad of Celite, washing the pad with methanol (20 mL) and ethyl acetate (20 mL). The solvents were removed under reduced pressure and the crude product was purified by chromatography on silica gel (30 g), eluting with 8% ethyl acetate in dichloromethane (4 L) to give 0.92 g (77%) of 23-d₉.

Step 4. 4-Amino-N-((2R,3S))-3-amino-2-hydroxy-4-phenylbutyl-N-(isobutyl-d₉)-benzenesulfonamide (24-d₉). To a solution of 23-d₉ (0.92 g, 1.84 mmol) in dichloromethane (20 mL) stirred at room temperature under nitrogen was added 4M hydrochloride solution in dioxane (1 mL, 4 mmol). Methanol (3 mL) was added and the resulting solution was stirred at room temperature under nitrogen for 3 hours. The solvents were removed under reduced pressure and the residue was dissolved in dichloromethane (20 mL). Water (10 mL) was added and the mixture was stirred in an ice-bath while 20% aqueous sodium hydroxide was slowly added to adjust the pH to 12. The phases were separated and the aqueous phase was extracted with dichloromethane (2×20 mL). The combined organic extracts were washed with brine (2×40 mL), dried over sodium sulfate and filtered. The solvent was removed under reduced pressure to give 0.71 g (96%) of 24-d₉ (a compound of Formula VII, wherein R¹ is —CD₂-CD-(CD₃)₂). ¹H-NMR (300 MHz, CDCl₃): δ 2.50 (dd, J=13.4, J₂=9.9, 1H), 2.97 (dd, J₁=13.2, J₂=3.8, 1H), 3.12-3.31 (m, 3H), 3.72-3.77 (m, 1H), 6.69 (d, J=8.8, 2H), 7.20-7.34 (m, 5H), 7.59 (d, J=8.8, 2H). HPLC (method: 20 mm C18-RP column—gradient method 2-95% ACN+0.1% formic acid in 3.3 min with 1.7 min hold at 95% ACN; Wavelength: 254 nm): retention time: 2.52 min. MS (M+H): 401.1.

### Example 2

### Synthesis of 2-(Methyl-d₃)-2,3,3,3-d₄-propan-1-amine (20-d₇)

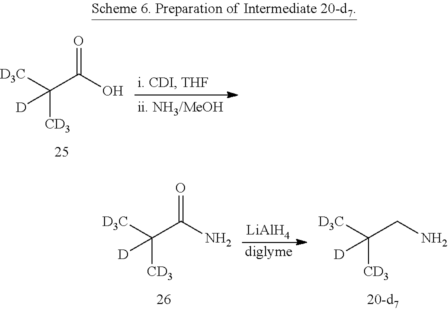

Step 1. (Isobutyr-d₇)-amide (26). To a suspension of carbonyl diimidazole (CDI, 17.0 g, 111 mmol, 1.01 equiv) in THF (220 mL) was added isobutyric-d₇-acid (25) (10.0 g, 105 mmol, 1 equiv, CDN Isotopes, 98 atom % D). The reaction was stirred at room temperature for 16 hours. A solution of 7 N NH₃ in MeOH (15 mL, 105 mmol, 1 equiv) was then added dropwise. During the addition, the reaction temperature reached 30° C. The reaction was stirred at room temperature for 3 hours. The solvent was evaporated under reduced pressure and the crude material was dissolved in THF (100 mL). 4 N HCl in dioxane (approximately 60 mL) was added and a white precipitate formed. The solids were removed by filtration and washed with THF. The filtrate was concentrated under reduced pressure and the crude product was purified by crystallization from EtOAc (30 mL) to afford 26 as a white solid (7.35 g, 76%).

Step 2. 2-(Methyl-d₃)-2,3,3,3-d₄-propan-1-amine (20-d₇). To a solution of 26 (7.00 g, 74 mmol, 1 equiv) in diglyme (250 mL), cooled to 0° C., was added in portions solid LiAlH₄ (3.11 g, 81 mmol, 1.1 equiv) keeping the reaction temperature below 10° C. The reaction mixture was allowed to slowly warm to room temperature and was stirred overnight. The resulting turbid solution was quenched by the addition of a saturated solution of Na₂SO₄ resulting in a thick, gray suspension. The suspension was filtered through a plug of Celite, washing with additional diglyme. The filtrate was subjected to short-path distillation at atmospheric pressure. The desired product began distilling when the head temperature reached 62° C. Distillation gave 20-d₇ as a clear, colorless liquid (2.51 g, 42%). GC analysis indicated this material was 70% pure.

### Example 3

Synthesis of 4-Amino-N-((2R,3S)-3-amino-2-hydroxy-4-phenylbutyl)-N-2-(methyl-d₃)-2,3,3,3-d₄-propyl-benzenesulfonamide (24-d₇). Intermediate 24-d₇ was prepared as generally outlined in Scheme 5 above with the exception that intermediate 20-d₉ was replaced with intermediate 20-d₇.

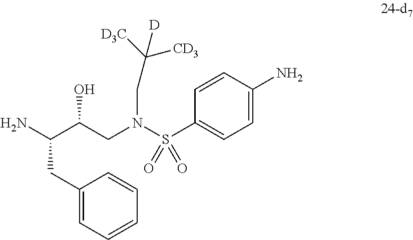

Step 1. tert-Butyl (2S,3R)-3-Hydroxy-4-(2-(methyl-d₃)-2,3,3,3-d₄-propyl-amino)-1-phenylbutan-2-ylcarbamate (21-d₇). Commercially-available tert-butyl (S)-1-((S)-oxiran-2-yl)-2-phenylethylcarbamate (10)(2.60 g, 9.8 mmol, 1 equiv) was suspended in isopropanol (60 mL). 20-d₇ (2.0 g, 17 mmol (assumed 70% pure, 1.7 equiv) was added and the resulting suspension was heated at reflux for 4 hours, then stirred at room temperature overnight. The solvent was then evaporated under reduced pressure to afford 21-d₇ as a white solid (3.10 g). LCMS indicated the purity of this material was 75%. The material was carried forward without further purification.

Step 2. tert-Butyl (2S,3R)-3-Hydroxy-4-(N-2-(methyl-d₃)-2,3,3,3-d₄-propyl-4-nitrophenylsulfonamido)-1-phenylbutan-2-ylcarbamate (22-d₇). Crude 21-d₇ (3.10 g, 7.4 mmol (assumed 75% purity), 1 equiv) was dissolved in CH₂Cl₂ (90 mL). Triethylamine (1.0 mL, 8.1 mmol, 1.1 equiv) was added followed by p-nitrobenzenesulfonyl chloride (1.62 g, 7.4 mmol, 1 equiv). The resulting solution was stirred at room temperature for 4 hours. The reaction was diluted with CH₂Cl₂ to a volume of 200 mL. The solution was washed with H₂O (150 mL), 0.5 N HCl (150 mL), H₂O (150 mL), and saturated aqueous NaHCO₃ (150 mL). The organic layer was dried over Na₂SO₄, filtered, and concentrated under reduced pressure to afford a pale yellow solid. The crude product was purified using an Analogix automated chromatography system eluting with CH₂Cl₂ for 10 minutes followed by a gradient of 0-4% MeOH/CH₂Cl₂ over 30 minutes. Fractions containing product were evaporated to give 22-d₇ as a white solid (3.03 g, 58% over two steps.)

Step 3. tert-Butyl(2S,3R)-4-(4-Amino-N-2-(methyl-d₃)-2,3,3,3-d₄-propyl-phenylsulfonamido)-3-hydroxy-1-phenylbutan-2-ylcarbamate (23-d₇). To a solution of 22-d₇ (1.40 g, 2.6 mmol, 1 equiv) in MeOH (30 mL) and EtOAc (30 mL) in a 500 mL Parr shaker bottle was added 20% Pd/C (50% wet, 0.20 g). The reaction mixture was shaken for 3 hours while maintaining a H₂ pressure of 35-40 psi. The reaction was filtered through a plug of Celite under a stream of nitrogen. The filtrate was concentrated in vacuo to afford a slightly yellow oil. The crude product was purified by silica gel chromatography eluting with 10% EtOAc/CH₂Cl₂. Fractions containing product were concentrated in vacuo to give 23-d₇ as a slightly yellow oil (1.30 g, 100%).

Step 3. 4-Amino-N-((2R,3S)-3-amino-2-hydroxy-4-phenylbutyl)-N-2-(methyl-d₃)-2,3,3,3-d₄-propyl-benzenesulfonamide (24-d₇). 4 N HCl in dioxane (8 mL, 32 mmol, 12 equiv) was added to a solution of 23-d₇ (1.30 g, 2.6 mmol, 1 equiv) in CH₂Cl₂ (25 mL). After approximately 5 minutes a white precipitate formed. MeOH (approximately 10 mL) was added until a clear solution was obtained. The reaction was then stirred at room temperature for 2 hours. The solvent was evaporated under reduced pressure and the crude material was partitioned between CH₂Cl₂ (50 mL) and H₂O (50 mL). The biphasic mixture was stirred vigorously and 10% NaOH was added until the pH reached 12. The mixture was transferred to a separatory funnel and the phases were separated. The aqueous layer was washed with an additional portion of CH₂Cl₂ (50 mL). The combined organic phases were washed with brine (2×200 mL), dried over Na₂SO₄, filtered, and concentrated under reduced pressure to give 24-d₇ as a slightly brown oil (0.88 g, 85%). The crude product was used without further purification.

### Example 4

### Synthesis of 2,5-Dioxopyrrolidin-1-yl (3R,3aS,6aR)-perdeuterofuro[2,3-b]furan-3-yl carbonate (35-d₉)

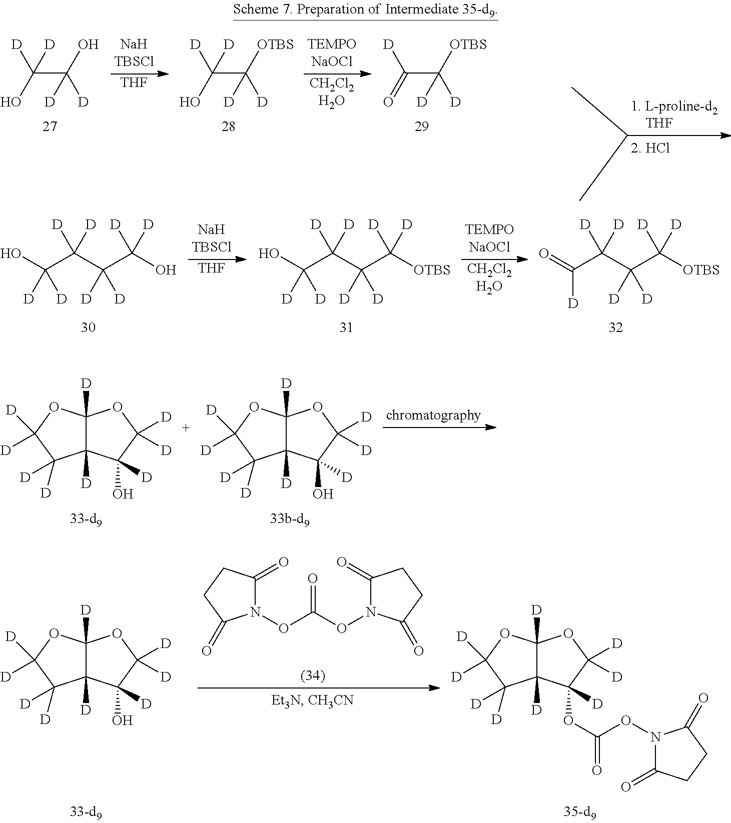

Step 1. 2-(tert-Butyldimethylsilyloxy)ethanol-d₄ (28). Sodium hydride (60% dispersion in mineral oil, 12.12 g, 303 mmol, 1 equiv) was placed in a 4-neck 1 L round-bottom flask and placed under N₂. The sodium hydride was washed with hexane (3×300 mL) to remove the mineral oil. After the final hexane wash, THF (500 mL) was added and the resulting suspension was cooled to 0° C. Ethylene glycol-d₄ (27) (20 g, 303 mmol, 1 equiv, Cambridge Isotope Labs, 98 atom % D) was added as a solution in THF (20 mL) all in one portion. A small exotherm was observed with the reaction temperature reaching 5° C. The reaction was stirred at 0° C. for 45 minutes during which time a white suspension formed. tert-Butyldimethylsilyl chloride (TBSCl, 45.7 g, 303 mmol, 1 equiv) was added in portions over 5 minutes. The reaction was stirred for 2.5 hours while warming to room temperature. The reaction was diluted with MTBE (400 mL) and washed with sat. aq. NaHCO₃ (2×1 L) and brine (500 mL). The organic layer was dried over Na₂SO₄, filtered, and evaporated under reduced pressure to afford 51 g of a clear, colorless liquid. The crude material was purified via silica gel chromatography eluting with 20% EtOAc in hexane. Fractions containing product were evaporated to give 28 as a clear, colorless liquid (42.1 g, 77%).

Step 2. 2-(tert-Butyldimethylsilyloxy)acetaldehyde-d₃ (29). A 2 L, 4-neck round-bottom flask was charged with 28 (35.6 g, 198 mmol, 1 equiv) and CH₂Cl₂ (400 mL). A solution of NaBr (2.3 g, 22 mmol, 0.11 equiv) in H₂O (12 mL) was added, followed by saturated aqueous NaHCO₃ (48 mL), and then by 2,2,6,6-tetramethylpiperidine 1-oxyl (TEMPO, 1.9 g, 12 mmol, 0.06 equiv). The resulting biphasic solution was cooled to 0° C. A 10-13% aqueous solution of NaOCl (144 mL) was diluted with H₂O (50 mL) and saturated aqueous NaHCO₃ (100 mL). This solution was added portion-wise (10-15 mL at a time) to the reaction with vigorous stirring. Upon addition, the color of the reaction became darker after several minutes and then faded. Once the color had faded, the next portion of NaOCl solution was added. The total time required for the addition of the NaOCl solution was 1.5 hours. Over the course of the reaction the temperature reached 18° C. After addition of the final portion of NaOCl the darker color persisted. The biphasic mixture was then transferred to a separatory funnel and the phases were separated. The organic layer was washed with saturated aqueous sodium thiosulfate (2×800 mL), dried over Na₂SO₄, filtered, and evaporated under reduced pressure to afford an orange liquid. The crude material was purified via Kugelrohr distillation (55° C. at 1.5 torr) to give 29 as an orange liquid (21.3 g, 61%). GC/MS indicated the material was 85% pure.

Step 3. 4-(tert-Butyldimethylsilyloxy)butan-d₄-1-ol (31). Sodium hydride (60% dispersion in mineral oil, 4.08 g, 102 mmol, 1 equiv) was placed in a 4-neck 1 L round-bottom flask and placed under N₂. The sodium hydride was washed with hexane (3×300 mL) to remove the mineral oil. After the final hexane wash, THF (350 mL) was added and the resulting suspension cooled to 0° C. 1,4-Butane-d₈-diol (30) (10 g, 102 mmol, 1 equiv, Aldrich, 98 atom % D) was added as a solution in THF (10 mL) all in one portion. A small exotherm was observed with the reaction temperature reaching 4° C. The reaction was stirred at 0° C. for 45 minutes during which time a white suspension formed. TBSCl (15.4 g, 102 mmol, 1 equiv) was added in portions over 5 minutes. The reaction was stirred for 2.5 hours while warming to room temperature. The reaction was diluted with MTBE (200 mL) and washed with saturated aqueous NaHCO₃ (2×500 mL) and brine (250 mL). The organic layer was dried over Na₂SO₄, filtered, and evaporated under reduced pressure to afford 23 g of a clear, colorless liquid. The crude material was purified via silica gel chromatography eluting with 20% EtOAc in hexane. Fractions containing product were concentrated to give 31 as a clear, colorless liquid (22.5 g, 100%).

Step 4. 4-(tert-Butyldimethylsilyloxy)butanal-d₇ (32). A 1 L, 4-neck round-bottom flask was charged with 31 (22.5 g, 102 mmol, 1 equiv) and CH₂Cl₂ (200 mL). A solution of NaBr (1.2 g, 11 mmol, 0.11 equiv) in H₂O (6 mL) was added, followed by saturated aqueous NaHCO₃ (24 mL), and then by TEMPO (0.40 g, 2.5 mmol, 0.03 equiv). The resulting biphasic solution was cooled to −5° C. using an ice-salt bath. A 10-13% aqueous solution of NaOCl (72 mL) was diluted with H₂O (25 mL) and saturated aqueous NaHCO₃ (50 mL). This solution was then added portion-wise (10 mL at a time) to the reaction with vigorous stirring. Upon addition the color of the reaction became darker after several minutes and then faded. Once the color had faded and the reaction temperature returned to 5° C., the next portion of NaOCl solution was added. The total time required for the addition of the NaOCl solution was 2 hours. Over the course of the reaction, the temperature was maintained below 10° C. After addition of the final portion of NaOCl the darker color persisted. The biphasic mixture was then transferred to a separatory funnel and the phases were separated. The organic layer was washed with saturated aqueous sodium thiosulfate (2×500 mL), dried over Na₂SO₄, filtered, and concentrated under reduced pressure to afford a slightly orange liquid. The crude material was purified via Kugelrohr distillation (70° C. at 1.2 torr) to give 32 as a slightly yellow liquid (15.1 g, 71%). GC/MS indicated this material was 92% pure.

Step 5. (3R,3aS,6aR)-Hexahydrofuro[2,3-b]furan-d₉-3-ol (33-d₉) and (3S,3aS,6aR)-hexahydrofuro[2,3-b]furan-d₉-3-ol (33b-d₉). To a cooled (4° C.) solution of 29 (5.0 g, 28.6 mmol, 2 equiv) and 32 (3.0 g, 14.4 mmol, 1 equiv) in THF (20 mL) was added L-proline-d₂ (420 mg, 3.6 mmol, 0.25 equiv; prepared by stirring L-proline in MeOD and evaporating the solvent two times). The reaction mixture was stirred 3 days at 4° C. A 3% aq. solution of HCl (5 mL) was then added and the reaction was stirred an additional 24 hours at 4° C. The reaction was warmed to room temperature and quenched by the addition of pyridine (0.75 mL), H₂O (10 mL), and toluene (40 mL). The resulting turbid biphasic mixture was stirred for 10 minutes and then filtered through a plug of Celite. The filtrate was transferred to a separatory funnel and the phases were separated. The organic layer was washed with additional H₂O (3×50 mL). The combined aqueous washes (including the initial H₂O) were washed with toluene (30 mL) and the combined organic layers were concentrated under reduced pressure. The residue was then co-evaporated with toluene (50 mL) resulting in crude 33-d₉/33b-d₉ as a yellow liquid (mixture of diastereomers by chiral GC, 3.6 g). The crude material was carried forward immediately without purification.

Step 6. (3R,3aS,6aR)-Hexahydrofuro[2,3-b]furan-d₉-3-ol (33-d₉). To a suspension of crude 33-d₉/33b-d₉ (3.6 g, 13.0 mmol—assuming 50% purity) in CH₂Cl₂ (50 mL) was added triethylamine (2 mL, 13.9 mmol. 1.1 equiv) and the mixture was stirred at room temperature for 20 minutes. Acetic anhydride (2.2 mL, 23.3 mmol, 1.8 equiv) was added as a solution in CH₂Cl₂ (5 mL) and the reaction was stirred for 5 hours. Additional amounts of triethylamine (2 mL) and acetic anhydride (2.2 mL) were added and the mixture was stirred overnight. Analysis showed only trace amounts of the desired product at this time. Silica gel was added directly to the mixture and solvent was evaporated under reduced pressure. The crude material absorbed onto silica gel was dry-loaded onto a silica gel column, eluting with a gradient of 30% EtOAc/heptanes to 60% EtOAc/heptanes. Several impurities eluted initially, followed by 33-d₉, the desired diastereomer. The column was then flushed with 10% MeOH/CH₂Cl₂ to elute 33b-d₉, the undesired diastereomer. Fractions containing 33-d₉ were concentrated in vacuo to afford a yellow liquid (600 mg). In addition, fractions containing 33b-d₉ were concentrated in vacuo to afford a yellow liquid (410 mg). Both samples were analyzed by chiral GC. 33-d₉ showed a single peak at 7.6 min, indicating a single enantiomer. 33b-d₉ showed a single peak at 8.1 minutes.

In an effort to confirm the stereochemistry of 33-d₉, a small portion was subjected to acetylation followed by stereoselective lipase cleavage conditions. 33-d₉ (70 mg, 0.5 mmol, 1 equiv) was suspended in CH₂Cl₂ (12 mL) and triethylamine (100 mg, 1.0 mmol, 2 equiv) and DMAP (10 mg, 0.08 mmol, 0.16 equiv) were added. The reaction was stirred for 15 minutes. Acetic anhydride (100 mg, 1 mmol, 2 equiv) was added and the reaction was stirred overnight. TLC analysis (heptanes/EtOAc 1:1) showed complete conversion to the acetate. The reaction was diluted with CH₂Cl₂ (5 mL) and extracted with 1:1 H₂O:brine (10 mL), 3% aq. HCl (10 mL), sat. aq. NaHCO₃ (10 mL), and brine (10 mL). The organic layer was dried over Na₂SO₄, filtered, and concentrated under reduced pressure giving a slightly yellow oil. The crude material was purified using an Analogix automated chromatography system eluting with a gradient of 10% EtOAc/heptanes to 40% EtOAc/heptanes over 30 minutes. All fractions were collected and analyzed by TLC as the product has no UV activity. Fractions containing product were concentrated to give the acetate of 33-d₉ as a slightly yellow oil (50 mg, 55%). Chiral GC analysis shows a single peak at 6.3 minutes.

To the acetate of 33-d₉ (50 mg, 0.28 mmol, 1 equiv) was added 1.5 mL of a NaH₂PO₄ buffer (pH=5.5), followed by 14 mg of Novozyme 435. The reaction was then heated to 45° C. After 2.5 hours, an aliquot was removed and concentrated. The residue was dissolved in MeOH (1 mL) and filtered through a syringe filter to remove insoluble salts. This sample was analyzed by chiral GC. The GC trace showed a single peak at 6.3 min, indicating no hydrolysis had occurred, thus confirming the stereochemistry of the acetate of 33-d₉ (and therefore 33-d₉).

Step 7. 2,5-Dioxopyrrolidin-1-yl(3S,3aR,6aS)-hexahydrofuro[2,3-b]furan-d₉-3-yl carbonate (35-d₉). Triethylamine (0.76 mL, 5.4 mmol, 1.8 equiv) was added to a solution of 33-d₉ (0.40 g, 2.9 mmol. 1 equiv) and bis(2,5-dioxopyrrolidin-1-yl) carbonate (34) (1.00 g, 3.9 mmol, 1.3 equiv) in acetonitrile (8 mL). The reaction was stirred at room temperature for 16 hours. The solvent was evaporated under reduced pressure and the residue was partitioned between EtOAc (100 mL) and sat. aq. NaHCO₃ (100 mL). The layers were separated and the aqueous layer was washed with additional EtOAc (50 mL). The combined organic layers were washed with brine (100 mL), dried over Na₂SO₄, filtered, and concentrated under reduced pressure to afford a slightly brown oil. The crude product was purified via silica gel chromatography eluting with a gradient of 0-2% MeOH/CH₂Cl₂. Fractions containing product were concentrated to give 35-d₉ as a colorless oil (0.40 g, 49%) and stored in the freezer to maintain stability.

### Example 5

### Synthesis of (3R,3aS,6aR)-Perdeuterofuro[2,3-b]furan-3-yl (2S,3R)-4-(4-amino-N-2-(methyl-d₃)-2,3,3,3-d₄-propyl-phenylsulfonamido)-3-hydroxy-1-phenylbutan-2-ylcarbamate (Compound 211)

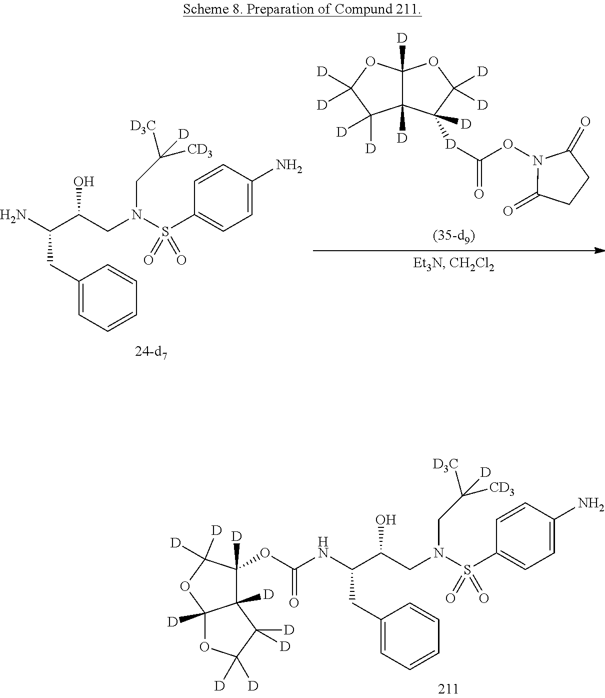

(3R,3aS,6aR)-Perdeuterofuro[2,3-b]furan-3-yl (2S,3R)-4-(4-amino-N-2-(methyl-d₃)-2,3,3,3-d₄-propyl-phenylsulfonamido)-3-hydroxy-1-phenylbutan-2-ylcarbamate (Compound 211). To a solution of 24-d₇ (80 mg, 0.20 mmol, 1 equiv) and 35-d₉ (60 mg, 0.21 mmol, 1.05 equiv) in CH₂Cl₂ (5 mL) was added triethylamine (40 mg, 0.4 mmol, 2 equiv). The solution was stirred for 6 hours, then was diluted with CH₂Cl₂ (10 mL) and washed with H₂O (20 mL) and brine (20 mL). The organic layer was dried over Na₂SO₄, filtered, and concentrated under reduced pressure to give a yellow oil. The crude material was purified using an Analogix automated chromatography system eluting with CH₂Cl₂ for 5 min followed by a gradient of 0-3% MeOH/CH₂Cl₂ over 40 minutes. Under these purification conditions significant amounts of the product decomposed, with the major decomposition being the displacement of the perdeuterated bis-THF moiety by methanol to form the methyl carbamate of 24-d₇. A portion of the desired compound did survive the above chromatography conditions. Fractions containing desired product were concentrated under reduced pressure and dried in a vacuum oven (40° C.) to give 211 as a clear, colorless film (13 mg, 12%). HPLC (method: 20 mm C18-RP column—gradient method 2-95% ACN+0.1% formic acid in 3.3 min with 1.7 min hold at 95% ACN; Wavelength: 254 nm): retention time: 3.33 min; 94.8% purity. MS (M+H): 564.3.

### Example 6

### Synthesis of (3R,3aS,6aR)-Perdeuterofuro[2,3-b]furan-3-yl (2S,3R)-4-(4-amino-N-(isobutyl-d₉)-phenylsulfonamido)-3-hydroxy-1-phenylbutan-2-ylcarbamate (Compound 267)

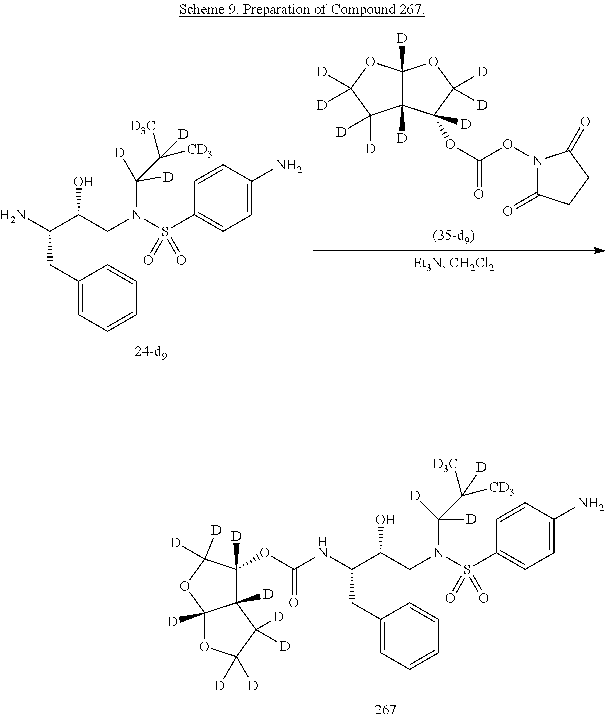

### (3R,3aS,6aR)-Perdeuterofuro[2,3-b]furan-3-yl (2S,3R)-4-(4-amino-N-(isobutyl-d₉)-phenylsulfonamido)-3-hydroxy-1-phenylbutan-2-ylcarbamate (Compound 267)

To a solution of 24-d₉ (25 mg, 0.06 mmol, 1 equiv) and 35-d₉ (20 mg, 0.07 mmol, 1.1 equiv) in CH₂Cl₂ (3 mL) was added triethylamine (13 mg, 0.12 mmol, 2 equiv). The solution was stirred for 4 hours. The reaction mixture was diluted with CH₂Cl₂ (10 mL) and washed with H₂O (20 mL) and brine (20 mL). The organic layer was dried over Na₂SO₄, filtered, and concentrated under reduced pressure to give a yellow film. The crude product was purified using an Analogix automated chromatography system eluting with a gradient of 30-100% EtOAc/heptanes over 40 minutes. These purification conditions removed several impurities but failed to provide pure 267. The recovered, impure 267 (15 mg) was resubjected to chromatography using an Analogix automated chromatography system eluting with CH₂Cl₂ for 2 minutes followed be a gradient of 0-5% IPA/CH₂Cl₂ over 40 minutes. These conditions provided improved separation of components with little decomposition being observed. Fractions containing product were concentrated under reduced pressure and dried in a vacuum oven (40° C.) to give 267 as a clear, colorless film (5 mg, 14%, 89% pure). MS (M+H): 566.4.

### Example 7

### Synthesis of (3R,3aS,6aR)-Perdeuterofuro[2,3-b]furan-3-yl (2S,3R)-4-(4-amino-N-isobutylphenylsulfonamido)-3-hydroxy-1-phenylbutan-2-ylcarbamate (Compound 155)

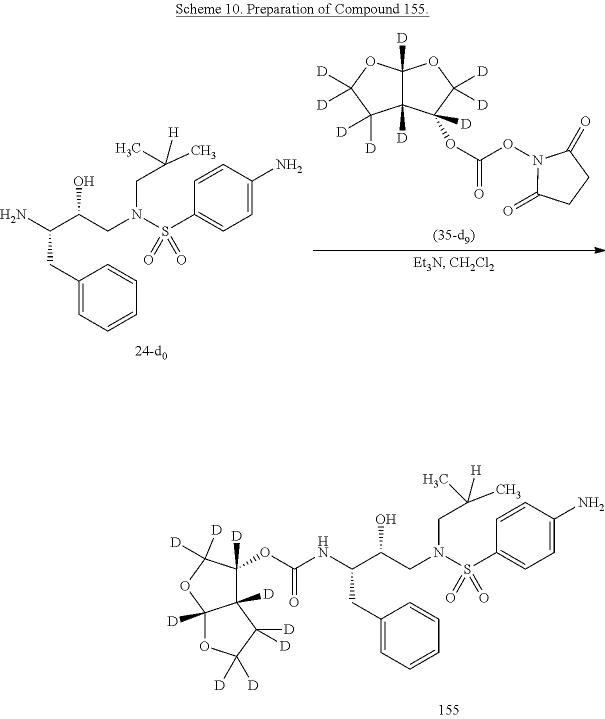

(3R,3aS,6aR)-Perdeuterofuro[2,3-b]furan-3-yl(2S,3R)-4-(4-amino-N-isobutylphenylsulfonamido)-3-hydroxy-1-phenylbutan-2-ylcarbamate (Compound 155). To a solution of 24-d₀ (25 mg, 0.06 mmol, 1 equiv, prepared according to the general methods of Ghosh, A K et al., J Org Chem 2004, 69, pp. 7822-7829) and 35-d₉ (20 mg, 0.07 mmol, 1.1 equiv) in CH₂Cl₂ (3 mL) was added triethylamine (13 mg, 0.12 mmol, 2 equiv). The solution was stirred for 4 hours. The reaction was diluted with CH₂Cl₂ (10 mL) and washed with H₂O (20 mL) and brine (20 mL). The organic layer was dried over Na₂SO₄, filtered, and concentrated under reduced pressure to give a yellow film. The crude product was purified using an Analogix automated chromatography system eluting with CH₂Cl₂ for 2 minutes followed by a gradient of 0-5% IPA/CH₂Cl₂ over 40 minutes. Fractions containing product were evaporated under reduced pressure and dried in a vacuum oven (40° C.) to give 155 as a clear, colorless film (6 mg, 18%, 89% pure). MS (M+H): 557.1.

### Example 8

### Synthesis of (3R,3aS,6aR)-6a-d₁-Hexahydrofuro[2,3-b]furan-3-ol (33-d₁)

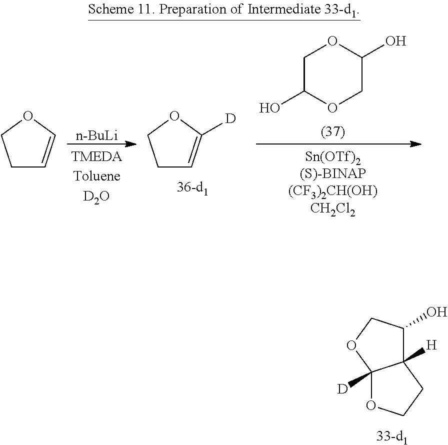

Synthesis of 5-d₁-2,3-dihyrofuran (36-d₁). To a flask containing neat 2,3-dihydrofuran (1.0 g, 14.27 mmol), was added N,N,N′,N′-tetramethylethylenediamine. To this solution was added n-butyl lithium (5.7 mL of a 2.5 M solution in toluene, 14.27 mmol) which resulted in the formation of an orange-colored precipitate. To this precipitate, was added deuterium oxide (2.0 mL) and the mixture stirred at ambient temperature for 30 minutes. The organic layer was separated, dried (MgSO₄), and filtered to afford a light yellow solution. The solution was fractionally distilled and the distillate that boiled from 50-55° C. was collected. This distillate collected contained the desired product 36-d₁ as a 2:1 mixture with toluene. ¹H NMR (CDCl₃, 400 MHz): δ 4.96 (t, J=4.8 Hz, 1H), 4.31 (t, J=19.2 Hz, 2H), 2.61 (dt, J1=9.6 Hz, J2=2.4 Hz, 2H).

Synthesis of (3R,3aS,6aR)-6a-d₁-hexahydrofuro[2,3-b]furan-3-ol (33-d₁). A mixture of (S)-BINAP (173 mg, 0.278 mmol) and tin triflate (101 mg, 0.242 mmol) in hexafluoroisopropanol-d (860 μL) and dichloromethane (2.00 mL) was stirred under nitrogen at room temperature for 40 minutes. To this mixture was added 1,4-dioxane-2,5-diol (37; 145 mg, 1.21 mmol) in dichloromethane (1.00 mL) and hexafluoroisopropanol-d (152 μL) and stirring was continued at room temperature for 30 minutes. The reaction mixture was cooled in an ice-bath and 36-d₁ (257 mg, 3.62 mmol) was slowly added as a 2:1 mixture in toluene. Stirring was continued in the ice-bath for 20 minutes then at room temperature overnight. The solvents were removed under reduced pressure to give crude 33-d₁ as a mixture of enantiomers. Purification on an ISCO automated chromatography instrument (4 g silica gel, 10 to 80% EtOAc in heptane) afforded 33-d₁ (204 mg, 64% yield) as a clear oil that was used without further purification in the next step. The enantiomeric ratio was not determined at this step. ¹H NMR (CDCl₃, 400 MHz): δ 4.50-4.38 (m, 1H), 4.02-3.94 (m, 2H), 3.94-3.84 (m, 1H), 3.66-3.58 (m, 1H), 2.85 (t, J=8.1 Hz, 1H), 2.35-2.26 (m, 1H), 2.19-1.98 (br s, 1H), 1.93-1.80 (m, 1H). MS (M+H): 132.2.

### Example 9

### Synthesis of (3R,3aS,6aR)-3,4,4,5,5-d₅-Hexahydrofuro[2,3-b]furan-3-ol (33-d_{5b})

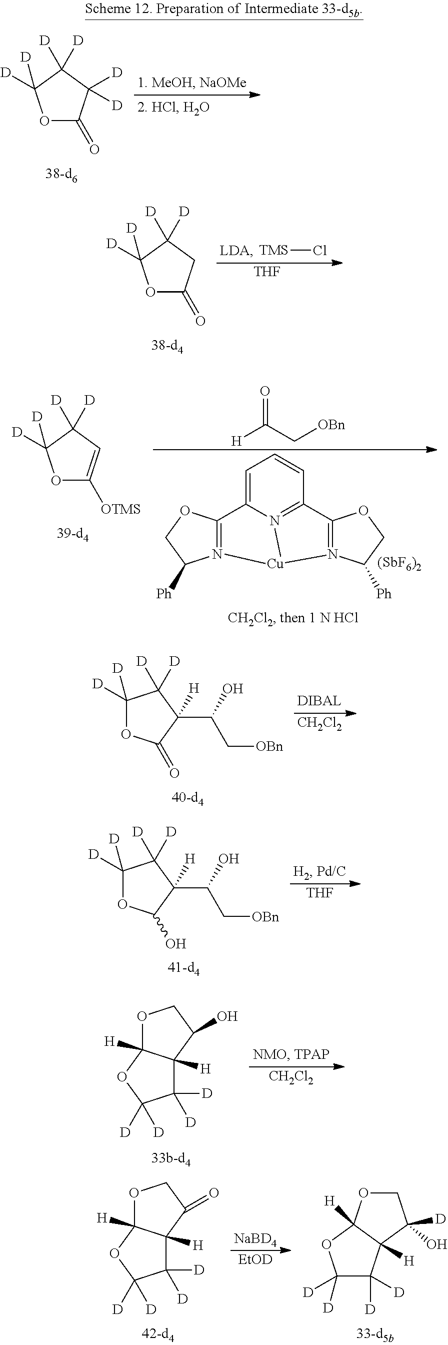

Step 1. 4,4,5,5-d₄-Dihydrofuran-2(3H)-one (38-d₄). Sodium metal (2.1 g, 90.6 mmol, 0.56 eq) was dissolved in MeOH (180 mL). To this was added γ-butyrolactone-d₆ (38-d₆) (15.0 g, 163 mmol, 1 eq; Aldrich, 98 atom % D) as a solution in MeOH (180 mL). The resulting solution was heated at reflux for 16 hours. The reaction was cooled to room temperature and concentrated under reduced pressure. A fresh portion of MeOH (360 mL) was added to the residue and the reaction was heated at reflux for an additional 16 hours. The reaction was cooled to room temperature and quenched by the addition of acetic acid (5.3 mL, 90.0 mmol) and several drops of concentrated HCl. The solvent was evaporated under reduced pressure and CH₂Cl₂ (100 mL) was added. The resulting suspension was filtered and the filtrate was evaporated under reduced pressure leaving a slightly yellow oil (γ-hydroxy-methylbutyrate-d₄). This material was dissolved in H₂O (100 mL) and concentrated HCl (9 mL) was added. The reaction was heated at reflux for 1 hour and then cooled to room temperature. The solution was saturated with NaCl and the product extracted with CH₂Cl₂ (3×500 mL). The combined organics were dried over Na₂SO₄, filtered, and evaporated to give 15.1 g of a yellow oil. The crude product was purified by Kugelrohr distillation yielding 13.9 g (94%) of 38-d₄ as a colorless liquid.

Step 2. Trimethyl(4,4,5,5-d₄-4,5-dihydrofuran-2-yloxy)silane (39-d₄). A solution of diisopropylamine (9.9 g, 98 mmol, 1.2 eq) in THF (89 mL) was cooled to 0° C. and a solution of n-butyllithium (36 mL, 2.5M in hexane, 90 mmol, 1.1 eq) was added dropwise over 10 minutes. The resulting yellow solution was stirred at 0° C. for 15 minutes and then cooled to −78° C. To this was added a solution of 38-d₄ (7.5 g, 83 mmol, 1 eq) and trimethylsilyl chloride (10.6 g, 98 mmol, 1.2 eq) in THF (56 mL) over 5 minutes. The reaction was warmed to room temperature and stirred for 2.5 hours. The majority of the THF was evaporated under reduced pressure and hexane was added to the remaining suspension. The solids were removed by filtration through a pad of Celite. The filtrate was concentrated under reduced pressure to a yellow oil. This material was purified by Kugelrohr distillation (bp 80° C. at 15 torr) giving 4.5 g (33%) of 39-d₄ as a colorless oil.

Step 3. (S)-3-((S)-2-(Benzyloxy)-1-hydroxyethyl)-4,4,5,5-d₄-dihydrofuran-2(3H)-one (40-d₄). A solution of [Cu((S,S)-Phenyl-bis(oxazolinyl)-pyridine)](SbF₆)₂ (0.0125M, 4.0 mL, 0.05 mmol, 0.05 eq; prepared according to JACS, 1996, 118, 5814) was cooled to −78° C. Benzyloxyacetaldehyde (0.14 mL, 1 mmol, 1 eq) and 39-d₄ (180 mg, 1.2 mmol, 1.2 eq) were added. The reaction was stirred 1 hour and then warmed to room temperature. The crude reaction mixture was filtered through a plug of silica gel washing with MTBE. The filtrate was concentrated under reduced pressure. The resulting oil was dissolved in THF (10 mL) and 1 N HCl (3 mL) was added. The mixture was stirred at room temperature for 15 minutes and then diluted with H₂O (15 mL). The aqueous mixture was extracted with MTBE (2×30 mL). The combined organic layers were washed with saturated aqueous NaHCO₃ (1×30 mL) and brine (1×30 mL), dried over Na₂SO₄, filtered, and concentrated to a slightly yellow oil. The crude material was purified using an Analogix automated chromatography system eluting with 30-60% ethyl acetate/heptanes. Fractions containing product were evaporated under reduced pressure yielding 150 mg (63%) of 40-d₄ as a colorless oil.

Step 4. (S)-3-((S)-2-(Benzyloxy)-1-hydroxyethyl)-4,4,5,5-d₄-tetrahydrofuran-2-ol (41-d₄). Compound 40-d₄ (150 mg, 0.63 mmol, 1 eq) was dissolved in CH₂Cl₂ (5 mL) and the solution cooled to −78° C. A solution of diisobutylaluminum hydride (1.6 mL, 1M in CH₂Cl₂, 2.5 eq) was added over a period of 5 minutes. The reaction was stirred for 45 minutes then quenched by the slow addition of MeOH (2 mL) and saturated aqueous potassium sodium tartrate (0.5 mL). The mixture was warmed to room temperature and stirred for 30 minutes. The turbid solution was filtered through Celite washing with CH₂Cl₂. The filtrate was concentrate giving 160 mg of 41-d₄ as a white solid which was used crude in the next step.

Step 5. (3S,3aS,6aR)-4,4,5,5-d₄-Hexahydrofuro[2,3-b]furan-3-ol (33b-d₄). To a solution of 41-d₄ (160 mg crude, 0.63 mmol (assumes 100% yield in previous step)) in THF (15 mL) was added 20% Pd/C, (50% wet, 20 mg). The resulting mixture was hydrogenated at 25 psi of H₂ for 30 minutes then was filtered through a pad of Celite under a stream of N₂ washing with THF. The filtrate was evaporated yielding 50 mg (61%) of 33b-d₄ as slightly yellow oil which was used without further purification in the next step.

Step 6. (3aR,6aR)-4,4,5,5-d₄-Tetrahydrofuro[2,3-b]furan-3(2H)-one (42-d₄). To a cold (0° C.) solution of 41-d₄ (50 mg, 0.37 mmol, 1 eq) in CH₂Cl₂ (10 mL) was added 4 Å molecular sieves (500 mg), tetrapropylammonium perruthenate (14 mg, 0.04 mmol, 0.1 eq), and 4-methylmorpholine-N-oxide (67 mg, 0.57 mmol, 1.5 eq). The brown/black reaction mixture was warmed to room temperature and stirred for 2 hours. The crude reaction mixture was then passed through a short plug of silica gel eluting with 50% ethyl acetate/heptane. The filtrate was concentrated yielding 28 mg (58%) of 42-d₄ as colorless oil which solidified upon standing.

Step 7. (3R,3aS,6aR)-3,4,4,5,5-d₅-Hexahydrofuro[2,3-b]furan-3-ol (33-d_{5b}). A solution of 42-d₄ (28 mg, 0.21 mmol, 1 eq) in EtOD (5 mL; 99.5 atom % D, Aldrich) was cooled to −15° C. Sodium borodeuteride (13 mg, 0.30 mmol, 1.4 eq; 99 atom % D, Cambridge Isotopes) was added in one portion and stirring was continued for 2.5 hours while allowing the solution to gradually warm to room temperature. The reaction was then quenched by the addition of saturated aqueous sodium chloride (2 mL). The mixture was extracted with EtOAc (3×50 mL). The combined organics were dried over Na₂SO₄, filtered, and concentrated to give 14 mg of crude 33-d_{5b} as light yellow oil. ¹H NMR (CDCl₃, 400 MHz): δ 5.69 (d, J=5.2, 1H), 3.99 (dd, J₁=9.2, J₂=0.9, 1H), 3.64 (d, J=9.1, 1H), 2.85-2.83 (m, 1H). MS (M+H): 136.2, (M+Na): 158.1.

### Example 10

### Synthesis of 2-(Benzyloxy)-2,2-d₂-acetaldehyde (44-d₂)

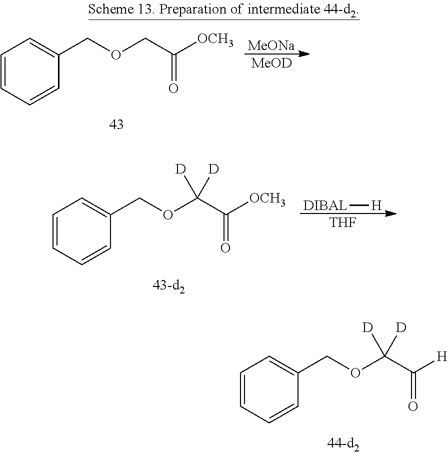

Step 1. Methyl 2-(benzyloxy)acetate-d₂ (43-d₂). Sodium metal (0.32 g, 13.8 mmol, 0.1 equiv) was dissolved in MeOD (150 mL; 99 atom % D, Cambridge Isotopes). To this was added a solution of 43 (25.0 g, 138 mmol, 1 equiv, prepared from commercially available methyl 2-(benzyloxy)acetate following a standard protocol of thionyl chloride in MeOH) in MeOD (100 mL). The resulting solution was heated to 40° C. and stirred for 24 hours. The solvent was evaporated and fresh MeOD (250 mL) was added. The resulting solution was again heated at 40° C. and stirred for 24 hours. This cycle was repeated a third time. After completion of the third cycle, the solvent was evaporated and the residue dissolved in EtOAc (300 mL). This solution was washed with D₂O (200 mL; 99 atom % D, Cambridge Isotopes) and brine (200 mL). The organic layer was dried over Na₂SO₄, filtered, and evaporated. The crude material was purified via silica gel chromatography eluting with 20% EtOAc/hexane. Fractions containing product were concentrated under reduced pressure giving 22.1 g (88%) of 43-d₂ as a clear colorless liquid.

Step 2. 2-(Benzyloxy)acetaldehyde-d₂ (44-d₂). A solution of 43-d₂ (1 g, 5.5 mmol) in CH₂Cl₂ (10 mL) was cooled to −78° C. and DIBAL (6 mL, 1M in CH₂Cl₂, 6 mmol, 1.1 eq) was added. After 2 hours additional DIBAL (1 mL, 1M in CH₂Cl₂) was added and the mixture was stirred at −78° C. for 1 hour. The reaction was quenched with water (10 mL), diluted with CH₂Cl₂ (50 mL), then saturated aqueous potassium sodium tartrate (100 mL) was added. The resulting mixture was allowed to warm to room temperature. The organic phase was separated and the aqueous phase was extracted by CH₂Cl₂ (3×50 mL). The combined organics were dried over Na₂SO₄, and concentrated to yield 44-d₂ as a clear oil (700 mg, 83.3%, 96% pure (GC)) which was used without purification.

### Example 11

### Synthesis of (3R,3aS,6aR)-2,2,3,4,4,5,5-d₇-Hexahydrofuro[2,3-b]furan-3-ol (33-d₇)

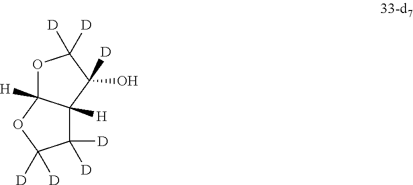

(3R,3aS,6aR)-2,2,3,4,4,5,5-d₇Hexahydrofuro[2,3-b]furan-3-ol (33-d₇). The synthesis of intermediate 33-d₇ was carried out in an analogous fashion to 33-d₅ (Example 9) from 39-d₄ with the exception that 2-(benzyloxy)acetaldehyde (step 3) was replaced with intermediate 44-d₂. ¹H NMR (CDCl₃, 400 MHz): δ 5.69 (d, J=5.3, 1H), 2.85-2.83 (m, 1H). MS (M+H): 138.0, (M+Na): 160.2.

### Example 12

### Synthesis of (3R,3aS,6aR)-2,2,4,4,5,5-d₆-Hexahydrofuro[2,3-b]furan-3-ol (33-d₆)

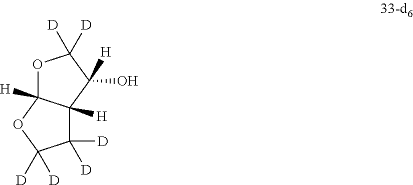

(3R,3aS,6aR)-2,2,4,4,5,5-d₆-Hexahydrofuro[2,3-b]furan-3-ol (33-d₆). The synthesis of intermediate 33-d₆ was carried out in an analogous fashion to 33-d₇ (Example 11) by reducing 42-d₆ with sodium borohydride in EtOD. ¹H NMR (CDCl₃, 400 MHz): δ 5.69 (d, J=5.1, 1H), 4.44 (d, J=4.5, 1H), 2.88-2.82 (m, 1H). MS (M+Na): 159.0.

### Example 13

Evaluation of Metabolic Stability in Human Liver Microsomes. Human liver microsomes (20 mg/mL) are available from Xenotech, LLC (Lenexa, Kans.). β-nicotinamide adenine dinucleotide phosphate, reduced form (NADPH), magnesium chloride (MgCl₂), and dimethyl sulfoxide (DMSO) are available from Sigma-Aldrich.

7.5 mM stock solutions of test compounds are prepared in DMSO. The 7.5 mM stock solutions are diluted to 50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. 10 μL of the 50 μM test compound is added to the microsomes and the mixture is pre-warmed for 10 minutes. Reactions arere initiated by addition of pre-warmed NADPH solution. The final reaction volume is 0.5 mL and contains 1 mg/mL human liver microsomes, 1 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contained 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer.

The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining(ln) vs incubation time]

Data analysis is performed using Microsoft Excel Software.

The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of Formula I: 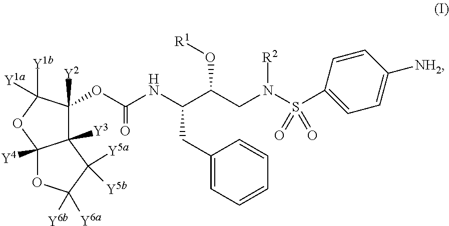
or a pharmaceutically acceptable salt thereof, wherein:
each Y is independently selected from hydrogen and deuterium;
at least one of Y⁴, Y^{5a}, Y^{5b}, Y^{6a} and Y^{6b} is deuterium;
R¹ is hydrogen or —(CR³R⁴—O)ₙ—R⁵;
R² is an isobutyl group having 0-9 deuterium;
R³ and R⁴ are independently selected from H and C₁-C₄ alkyl;
R⁵ is selected from an α-amino acid, —C(O)R⁶, —P(O)—(OM)₂ and —S(O)—OM;
R⁶ is hydrogen or an optionally substituted C₁-C₇ alkyl;
each M is H, or a cation independently selected from Li⁺, Na⁺, K⁺, Mg²⁺, Ca²⁺, Ba²⁺, and NH₄⁺; and
n is 0 or 1.

2. The compound of claim 1 wherein R¹ is hydrogen or —(CH₂—O)ₙ—R⁵.

3. The compound of claim 2 wherein R⁵ is an α-amino acid with either the (D)-, (L)-, or racemic (D,L) configuration.

4. The compound of claim 3 wherein R⁵ is an α-amino acid having an (L)-configuration and is selected from serine, lysine, tyrosine, valine, glutamic acid, aspartic acid, 3-pyridylalanine and histidine.

5. The compound of claim 2 wherein R⁵ is —C(O)—R⁶, and R⁶ is a C₁-C₇ alkyl, wherein R⁶ is optionally substituted with halo, cyano, hydroxyl, carboxy, alkoxy, oxo, amino, alkylamino, dialkylamino, cycloheteroalkyl, aryl, arylalkyl, heteroaryl, or heteroarylalkyl, and wherein any cyclic portion of the substituent of R⁶ is further optionally substituted.

6. The compound of claim 5 wherein R⁶ is selected from: —CH₂OCH₃; —CH₂CH₂OCH₃; —CH₂CH₂CO₂H; —CH₂CH₂NH₂; —CH₂CH₂NH—CH₃; —CH₂CH₂N(CH₃)₂;

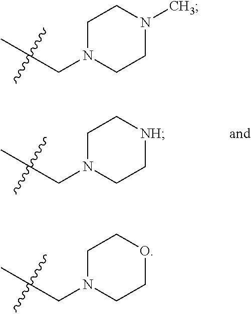

7. The compound of claim 2 wherein R⁵ is —P(O)—(OM)₂ or —S(O)—OM, and M is a cation selected from Na⁺, Mg²⁺ and NH₄⁺.

8. The compound of claim 7 wherein M is Na⁺.

9. The compound of claim 1 wherein R¹ is hydrogen.

10. The compound of claim 1, wherein R² is selected from —CH₂CH(CH₃)₂, —CH₂CH(CD₃)₂, —CH₂CD(CH₃)₂, —CD₂CH(CH₃)₂, —CH₂CD(CD₃)₂, —CD₂CH(CD₃)₂, —CD₂CD(CH₃)₂, and —CD₂CD(CD₃)₂.

11. The compound of claim 10, wherein R² is selected from —CH₂CH(CH₃)₂, —CH₂CD(CH₃)₂, —CD₂CH(CH₃)₂, —CH₂CD(CD₃)₂, —CD₂CD(CH₃)₂, and —CD₂CD(CD₃)₂.

12. The compound of claim 11, wherein R² is selected from —CH₂CH(CH₃)₂, —CH₂CD(CD₃)₂ and —CD₂CD(CD₃)₂.

13. The compound of claim 1, wherein Y^{1a} and Y^{1b} are the same, Y^{5a} and Y^{5b} are the same, and Y^{6a} and Y^{6b} are the same.

14. The compound of claim 13 having the formula: 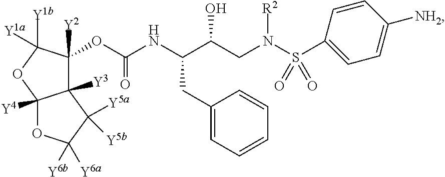
wherein the compound is selected from any one of the compounds set forth in the table below or a pharmaceutically acceptable salt thereof:

| Cmpd # | R² | each Y¹ | Y² | Y³ | Y⁴ | each Y⁵ | each Y⁶ |
|---|---|---|---|---|---|---|---|
| 100 | CH₂CH(CH₃)₂ | H | H | H | H | H | D |
| 101 | CH₂CH(CH₃)₂ | H | H | H | H | D | H |
| 102 | CH₂CH(CH₃)₂ | H | H | H | D | H | H |
| 103 | CH₂CH(CH₃)₂ | H | H | H | H | D | D |
| 104 | CH₂CH(CH₃)₂ | H | H | H | D | D | H |
| 105 | CH₂CH(CH₃)₂ | H | H | H | D | H | D |
| 106 | CH₂CH(CH₃)₂ | H | H | H | D | D | D |
| 107 | CH₂CH(CH₃)₂ | H | H | D | H | H | D |
| 108 | CH₂CH(CH₃)₂ | H | H | D | H | D | H |
| 109 | CH₂CH(CH₃)₂ | H | H | D | D | H | H |
| 110 | CH₂CH(CH₃)₂ | H | H | D | H | D | D |
| 111 | CH₂CH(CH₃)₂ | H | H | D | D | D | H |
| 112 | CH₂CH(CH₃)₂ | H | H | D | D | H | D |
| 113 | CH₂CH(CH₃)₂ | H | H | D | D | D | D |
| 114 | CH₂CH(CH₃)₂ | H | D | H | H | H | D |
| 115 | CH₂CH(CH₃)₂ | H | D | H | H | D | H |
| 116 | CH₂CH(CH₃)₂ | H | D | H | D | H | H |
| 117 | CH₂CH(CH₃)₂ | H | D | H | H | D | D |
| 118 | CH₂CH(CH₃)₂ | H | D | H | D | D | H |
| 119 | CH₂CH(CH₃)₂ | H | D | H | D | H | D |
| 120 | CH₂CH(CH₃)₂ | H | D | H | D | D | D |
| 121 | CH₂CH(CH₃)₂ | D | H | H | H | H | D |
| 122 | CH₂CH(CH₃)₂ | D | H | H | H | D | H |
| 123 | CH₂CH(CH₃)₂ | D | H | H | D | H | H |
| 124 | CH₂CH(CH₃)₂ | D | H | H | H | D | D |
| 125 | CH₂CH(CH₃)₂ | D | H | H | D | D | H |
| 126 | CH₂CH(CH₃)₂ | D | H | H | D | H | D |
| 127 | CH₂CH(CH₃)₂ | D | H | H | D | D | D |
| 128 | CH₂CH(CH₃)₂ | H | D | D | H | H | D |
| 129 | CH₂CH(CH₃)₂ | H | D | D | H | D | H |
| 130 | CH₂CH(CH₃)₂ | H | D | D | D | H | H |
| 131 | CH₂CH(CH₃)₂ | H | D | D | H | D | D |
| 132 | CH₂CH(CH₃)₂ | H | D | D | D | D | H |
| 133 | CH₂CH(CH₃)₂ | H | D | D | D | H | D |
| 134 | CH₂CH(CH₃)₂ | H | D | D | D | D | D |
| 135 | CH₂CH(CH₃)₂ | D | D | H | H | H | D |
| 136 | CH₂CH(CH₃)₂ | D | D | H | H | D | H |
| 137 | CH₂CH(CH₃)₂ | D | D | H | D | H | H |
| 138 | CH₂CH(CH₃)₂ | D | D | H | H | D | D |
| 139 | CH₂CH(CH₃)₂ | D | D | H | D | D | H |
| 140 | CH₂CH(CH₃)₂ | D | D | H | D | H | D |
| 141 | CH₂CH(CH₃)₂ | D | D | H | D | D | D |
| 142 | CH₂CH(CH₃)₂ | D | H | D | H | H | D |
| 143 | CH₂CH(CH₃)₂ | D | H | D | H | D | H |
| 144 | CH₂CH(CH₃)₂ | D | H | D | D | H | H |
| 145 | CH₂CH(CH₃)₂ | D | H | D | H | D | D |
| 146 | CH₂CH(CH₃)₂ | D | H | D | D | D | H |
| 147 | CH₂CH(CH₃)₂ | D | H | D | D | H | D |
| 148 | CH₂CH(CH₃)₂ | D | H | D | D | D | D |
| 149 | CH₂CH(CH₃)₂ | D | D | D | H | H | D |
| 150 | CH₂CH(CH₃)₂ | D | D | D | H | D | H |
| 151 | CH₂CH(CH₃)₂ | D | D | D | D | H | H |
| 152 | CH₂CH(CH₃)₂ | D | D | D | H | D | D |
| 153 | CH₂CH(CH₃)₂ | D | D | D | D | D | H |
| 154 | CH₂CH(CH₃)₂ | D | D | D | D | H | D |
| 155 | CH₂CH(CH₃)₂ | D | D | D | D | D | D |
| 156 | CH₂CD(CD₃)₂ | H | H | H | H | H | D |
| 157 | CH₂CD(CD₃)₂ | H | H | H | H | D | H |
| 158 | CH₂CD(CD₃)₂ | H | H | H | D | H | H |
| 159 | CH₂CD(CD₃)₂ | H | H | H | H | D | D |
| 160 | CH₂CD(CD₃)₂ | H | H | H | D | D | H |
| 161 | CH₂CD(CD₃)₂ | H | H | H | D | H | D |
| 162 | CH₂CD(CD₃)₂ | H | H | H | D | D | D |
| 163 | CH₂CD(CD₃)₂ | H | H | D | H | H | D |
| 164 | CH₂CD(CD₃)₂ | H | H | D | H | D | H |
| 165 | CH₂CD(CD₃)₂ | H | H | D | D | H | H |
| 166 | CH₂CD(CD₃)₂ | H | H | D | H | D | D |
| 167 | CH₂CD(CD₃)₂ | H | H | D | D | D | H |
| 168 | CH₂CD(CD₃)₂ | H | H | D | D | H | D |
| 169 | CH₂CD(CD₃)₂ | H | H | D | D | D | D |
| 170 | CH₂CD(CD₃)₂ | H | D | H | H | H | D |
| 171 | CH₂CD(CD₃)₂ | H | D | H | H | D | H |
| 172 | CH₂CD(CD₃)₂ | H | D | H | D | H | H |
| 173 | CH₂CD(CD₃)₂ | H | D | H | H | D | D |
| 174 | CH₂CD(CD₃)₂ | H | D | H | D | D | H |
| 175 | CH₂CD(CD₃)₂ | H | D | H | D | H | D |
| 176 | CH₂CD(CD₃)₂ | H | D | H | D | D | D |
| 177 | CH₂CD(CD₃)₂ | D | H | H | H | H | D |
| 178 | CH₂CD(CD₃)₂ | D | H | H | H | D | H |
| 179 | CH₂CD(CD₃)₂ | D | H | H | D | H | H |
| 180 | CH₂CD(CD₃)₂ | D | H | H | H | D | D |
| 181 | CH₂CD(CD₃)₂ | D | H | H | D | D | H |
| 182 | CH₂CD(CD₃)₂ | D | H | H | D | H | D |
| 183 | CH₂CD(CD₃)₂ | D | H | H | D | D | D |
| 184 | CH₂CD(CD₃)₂ | H | D | D | H | H | D |
| 185 | CH₂CD(CD₃)₂ | H | D | D | H | D | H |
| 186 | CH₂CD(CD₃)₂ | H | D | D | D | H | H |
| 187 | CH₂CD(CD₃)₂ | H | D | D | H | D | D |
| 188 | CH₂CD(CD₃)₂ | H | D | D | D | D | H |
| 189 | CH₂CD(CD₃)₂ | H | D | D | D | H | D |
| 190 | CH₂CD(CD₃)₂ | H | D | D | D | D | D |
| 191 | CH₂CD(CD₃)₂ | D | D | H | H | H | D |
| 192 | CH₂CD(CD₃)₂ | D | D | H | H | D | H |
| 193 | CH₂CD(CD₃)₂ | D | D | H | D | H | H |
| 194 | CH₂CD(CD₃)₂ | D | D | H | H | D | D |
| 195 | CH₂CD(CD₃)₂ | D | D | H | D | D | H |
| 196 | CH₂CD(CD₃)₂ | D | D | H | D | H | D |
| 197 | CH₂CD(CD₃)₂ | D | D | H | D | D | D |
| 198 | CH₂CD(CD₃)₂ | D | H | D | H | H | D |
| 199 | CH₂CD(CD₃)₂ | D | H | D | H | D | H |
| 200 | CH₂CD(CD₃)₂ | D | H | D | D | H | H |
| 201 | CH₂CD(CD₃)₂ | D | H | D | H | D | D |
| 202 | CH₂CD(CD₃)₂ | D | H | D | D | D | H |
| 203 | CH₂CD(CD₃)₂ | D | H | D | D | H | D |
| 204 | CH₂CD(CD₃)₂ | D | H | D | D | D | D |
| 205 | CH₂CD(CD₃)₂ | D | D | D | H | H | D |
| 206 | CH₂CD(CD₃)₂ | D | D | D | H | D | H |
| 207 | CH₂CD(CD₃)₂ | D | D | D | D | H | H |
| 208 | CH₂CD(CD₃)₂ | D | D | D | H | D | D |
| 209 | CH₂CD(CD₃)₂ | D | D | D | D | D | H |
| 210 | CH₂CD(CD₃)₂ | D | D | D | D | H | D |
| 211 | CH₂CD(CD₃)₂ | D | D | D | D | D | D |
| 212 | CD₂CD(CD₃)₂ | H | H | H | H | H | D |
| 213 | CD₂CD(CD₃)₂ | H | H | H | H | D | H |
| 214 | CD₂CD(CD₃)₂ | H | H | H | D | H | H |
| 215 | CD₂CD(CD₃)₂ | H | H | H | H | D | D |
| 216 | CD₂CD(CD₃)₂ | H | H | H | D | D | H |
| 217 | CD₂CD(CD₃)₂ | H | H | H | D | H | D |
| 218 | CD₂CD(CD₃)₂ | H | H | H | D | D | D |
| 219 | CD₂CD(CD₃)₂ | H | H | D | H | H | D |
| 220 | CD₂CD(CD₃)₂ | H | H | D | H | D | H |
| 221 | CD₂CD(CD₃)₂ | H | H | D | D | H | H |
| 222 | CD₂CD(CD₃)₂ | H | H | D | H | D | D |
| 223 | CD₂CD(CD₃)₂ | H | H | D | D | D | H |
| 224 | CD₂CD(CD₃)₂ | H | H | D | D | H | D |
| 225 | CD₂CD(CD₃)₂ | H | H | D | D | D | D |
| 226 | CD₂CD(CD₃)₂ | H | D | H | H | H | D |
| 227 | CD₂CD(CD₃)₂ | H | D | H | H | D | H |
| 228 | CD₂CD(CD₃)₂ | H | D | H | D | H | H |
| 229 | CD₂CD(CD₃)₂ | H | D | H | H | D | D |
| 230 | CD₂CD(CD₃)₂ | H | D | H | D | D | H |
| 231 | CD₂CD(CD₃)₂ | H | D | H | D | H | D |
| 232 | CD₂CD(CD₃)₂ | H | D | H | D | D | D |
| 233 | CD₂CD(CD₃)₂ | D | H | H | H | H | D |
| 234 | CD₂CD(CD₃)₂ | D | H | H | H | D | H |
| 235 | CD₂CD(CD₃)₂ | D | H | H | D | H | H |
| 236 | CD₂CD(CD₃)₂ | D | H | H | H | D | D |
| 237 | CD₂CD(CD₃)₂ | D | H | H | D | D | H |
| 238 | CD₂CD(CD₃)₂ | D | H | H | D | H | D |
| 239 | CD₂CD(CD₃)₂ | D | H | H | D | D | D |
| 240 | CD₂CD(CD₃)₂ | H | D | D | H | H | D |
| 241 | CD₂CD(CD₃)₂ | H | D | D | H | D | H |
| 242 | CD₂CD(CD₃)₂ | H | D | D | D | H | H |
| 243 | CD₂CD(CD₃)₂ | H | D | D | H | D | D |
| 244 | CD₂CD(CD₃)₂ | H | D | D | D | D | H |
| 245 | CD₂CD(CD₃)₂ | H | D | D | D | H | D |
| 246 | CD₂CD(CD₃)₂ | H | D | D | D | D | D |
| 247 | CD₂CD(CD₃)₂ | D | D | H | H | H | D |
| 248 | CD₂CD(CD₃)₂ | D | D | H | H | D | H |
| 249 | CD₂CD(CD₃)₂ | D | D | H | D | H | H |
| 250 | CD₂CD(CD₃)₂ | D | D | H | H | D | D |
| 251 | CD₂CD(CD₃)₂ | D | D | H | D | D | H |
| 252 | CD₂CD(CD₃)₂ | D | D | H | D | H | D |
| 253 | CD₂CD(CD₃)₂ | D | D | H | D | D | D |
| 254 | CD₂CD(CD₃)₂ | D | H | D | H | H | D |
| 255 | CD₂CD(CD₃)₂ | D | H | D | H | D | H |
| 256 | CD₂CD(CD₃)₂ | D | H | D | D | H | H |
| 257 | CD₂CD(CD₃)₂ | D | H | D | H | D | D |
| 258 | CD₂CD(CD₃)₂ | D | H | D | D | D | H |
| 259 | CD₂CD(CD₃)₂ | D | H | D | D | H | D |
| 260 | CD₂CD(CD₃)₂ | D | H | D | D | D | D |
| 261 | CD₂CD(CD₃)₂ | D | D | D | H | H | D |
| 262 | CD₂CD(CD₃)₂ | D | D | D | H | D | H |
| 263 | CD₂CD(CD₃)₂ | D | D | D | D | H | H |
| 264 | CD₂CD(CD₃)₂ | D | D | D | H | D | D |
| 265 | CD₂CD(CD₃)₂ | D | D | D | D | D | H |
| 266 | CD₂CD(CD₃)₂ | D | D | D | D | H | D |
| and 267 | CD₂CD(CD₃)₂ | D | D | D | D | D | D. |

15. The compound of claim 1 selected from any one of: 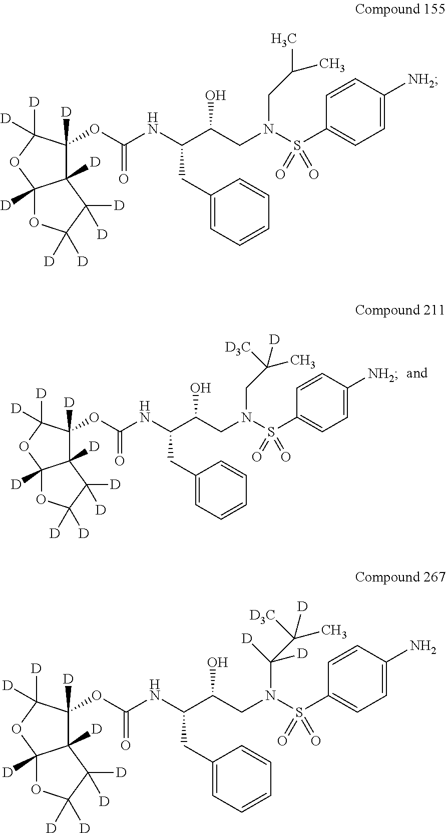
or a pharmaceutically acceptable salt thereof.

16. The compound of claim 1 selected from any one of: 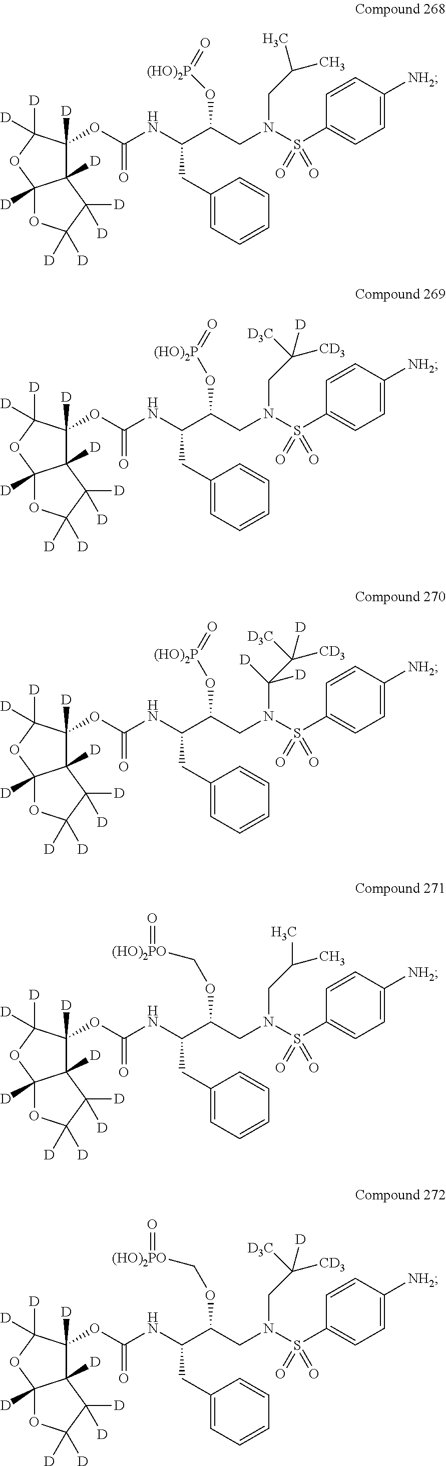 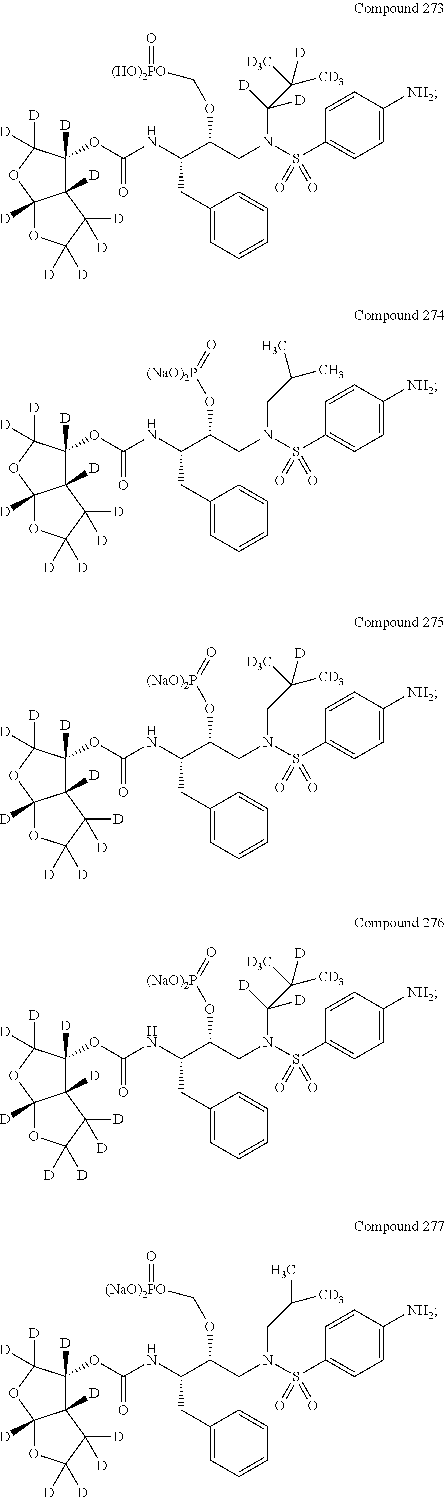 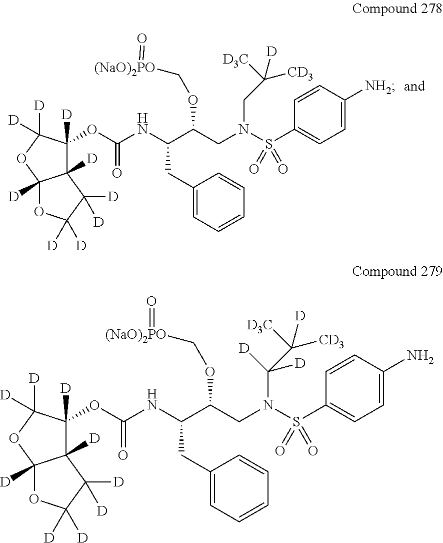
or a pharmaceutically acceptable salt of any of the foregoing.

17. The compound of claim 1, wherein the compound is a compound of the Formula Ia: 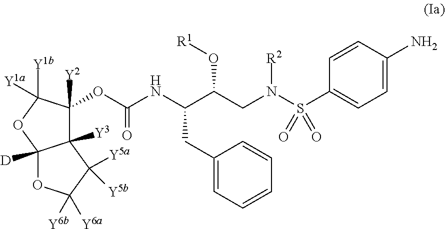
or a pharmaceutically acceptable salt thereof.

18. The compound of claim 1, wherein the compound is a compound of the Formula Ib: 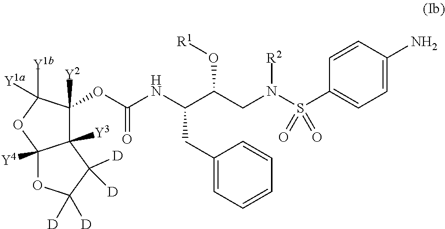
or a pharmaceutically acceptable salt thereof.

19. A compound of Formula IIa: 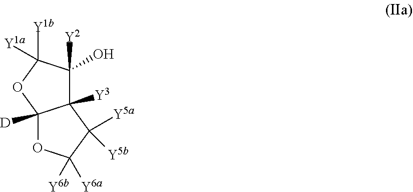
or a salt thereof,
wherein: each Y is independently selected from hydrogen and deuterium.

20. The compound of claim 19 selected from any one of: 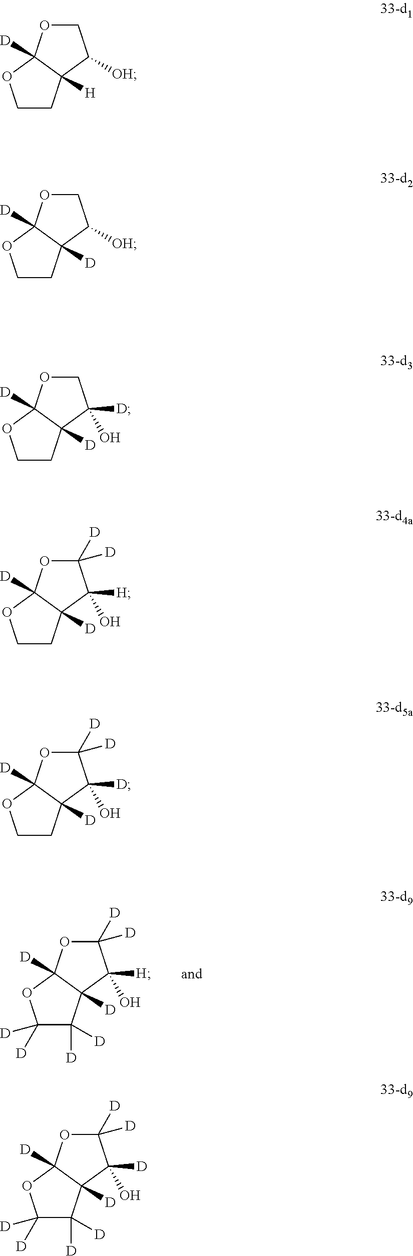
or a salt thereof.

21. A compound of Formula IIb: 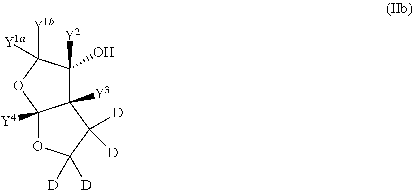
or a salt thereof,
wherein: each Y is independently selected from hydrogen and deuterium.

22. The compound of claim 21 selected from any one of: 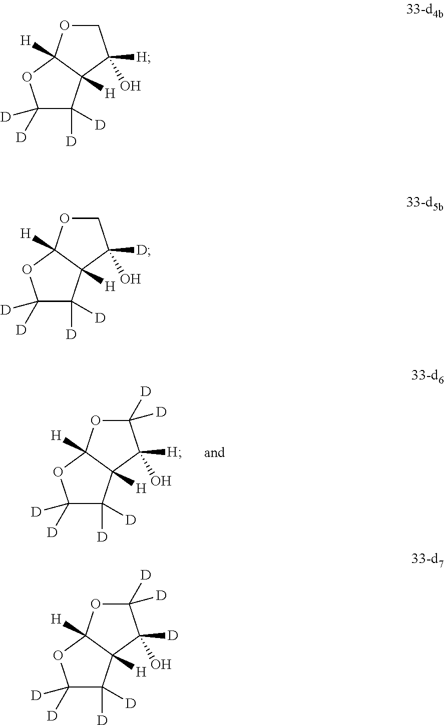
or a salt thereof.

23. The compound of claim 1, wherein any atom not designated as deuterium is present at its natural isotopic abundance.

24. A pharmaceutical composition comprising a compound of claim 1, and a pharmaceutically acceptable carrier.

25. The composition of claim 24, additionally comprising a second therapeutic agent useful in the treatment of HIV infection or malaria.

26. The composition of claim 25, wherein the second therapeutic agent is selected from ritonavir, atazanavir, indinavir, etravirine, tenofovir, emtricitabine, zidovudine, lopinavir, efavirenz, fosamprenavir, tipranavir, nevirapine, lamivudine, abacavir and combinations thereof.

27. A method of treating a disease or condition selected from HIV infection and malaria in a patient in need thereof comprising administering to the patient an effective amount of a composition of claim 24.

28. A method of claim 27, wherein the disease or condition is HIV infection.

29. A method of claim 28, further comprising administering to the patient in need thereof a second therapeutic agent useful in the treatment of HIV infection.

30. A method of claim 29, wherein the second therapeutic agent is selected from ritonavir, atazanavir, indinavir, etravirine, tenofovir, emtricitabine, zidovudine, lopinavir, efavirenz, fosamprenavir, tipranavir, nevirapine, lamivudine, abacavir and combinations thereof.
